(19) United States
(12) Patent Application Publication
(10) Pub. No.: US 2005/0009886 A1
(43) Pub. Date: Jan. 13, 2005

(54) 1,4-Dihydropyridine derivative with a guaiacoxypropanolamine and/or phenoxypropanolamine moiety

(76) Inventor: Ing-Jun Chen, Kaohsiung (TW)

Correspondence Address: BIRCH STEWART KOLASCH & BIRCH, PO BOX 747, FALLS CHURCH, VA, 22040-0747, US

(21) Appl. No.: 10/851,293

(22) Filed: May 24, 2004

Related U.S. Application Data
(63) Continuation-in-part of application No. 10/119,709, filed on Apr. 11, 2002, now Pat. No. 6,794,399.
Application No. 10/119,709 is a continuation of application No. 09/347,763, filed on Jul. 6, 1999, now abandoned.

(30) Foreign Application Priority Data
Jul. 21, 1998 (CN) 87111827

(51) Int. Cl. A61K 31/455; C07D 213/46

(52) U.S. Cl. 514/355; 546/315

(57) ABSTRACT

The invention pertains to a compound of the following formulas: 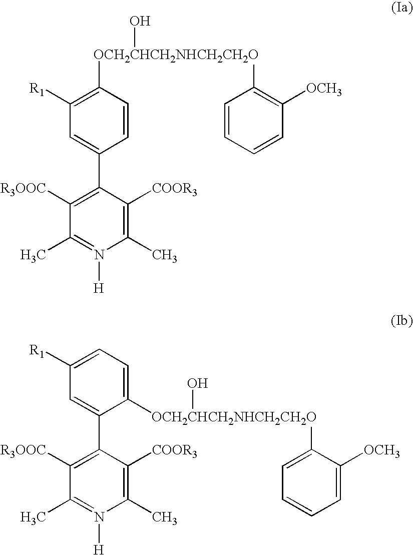
wherein R₁ and R₃ are each individually selected from the group consisting of —X, —H, —NO₂, CF₃, saturated C₁-C₆ alkyl chain, unsaturated C₂-C₆ alkyl chain, saturated C₁-C₆ alkoxy chain, and unsaturated C₂-C₆ alkoxy chain, and wherein X represents a halogen. The inventive compounds exhibit pharmacological activity for blocking an α-, β-adrenoreceptor or calcium channel, inducing hypotension or inducing vaso-relaxation.

This application is a continuation-in-part of application Ser. No. 10/119,709, filed Apr. 11, 2002, which is a continuation of application Ser. No. 09/347,763, filed Jul. 6, 1999.

This application claims priority of China 87111827, filed Jul. 21, 1998, the entire contents of which are hereby incorporated by reference.

### BACKGROUND OF THE INVENTION

1. Field of the Invention

The invention pertains to the continuously maintenance of hypotension, with activation of a competitive β-adrenoreceptor and calcium ion blocking agent, an induced vasorelaxing effect, with the activation of calcium ion channel antagonist and β-adrenoreceptor antagonist, and particularly to a 1,4-dihydropiridine derivative chemically bound with a guaiacoxypropanolamine and/or phenoxypropanolamine moiety.

2. Description of the Related Art

Serious and pernicious hypertensive subjects could obtain rapid hypotension by treatment with nifedipine, yet too rapid or strong effect could result in tachycardia. In later experiments, it is found that while administrating vasodilator, provision of β-adrenoceptor blocking agent to subjects could inhibit tachycardia induced by sympathetic excitation. Clinical reports have shown that in the treatment of angina pectoris and hypertension, combination therapy of β-adrenoceptor blocking agent and calcium entry blocking agent has advantage efficacy over any one of those drugs (Fitzsimons, T. J., J Hypertens., 5, pp. S11-S15, 1987).

In most original hypertension subjects, their peripheral circulation usually has a much higher resistance than normal. Though a direct vasodilator could reduce the resistance, therefore these drugs could induce unwanted effects, including reflex tachycardia due to baroreceptor activation which may impair the hypotensive effect by blood vessel contraction; tachycardia; increase of cardiac output. Some reports have already pointed out that β-adrenoceptor blocking agent could inhibit tachycardia due to sympathetic excitation after administration of vasodilator.

Nifedipine is a peripheral vasodilator with 1,4-dihydropiridine ring. It is effective on the cardiac blood vessels, including vasodilatation, where it functions by directly inhibit the result of calcium ion inflow in vessel's smooth muscle membrane. Theoretically, combination therapy of calcium entry blocking agent and β-adrenoceptor blocking agent may result in two different cardiac suppression effect. Nevertheless, reports have demonstrated that though nifedipine would increase the heart rate and renin level in hypertensive subjects, these effects could be inhibited by concurrent administration of propranolol. Furthermore, it has been observed that there is coordinating effect produced in the blood pressure, which lower the pressure even further.

To overcome the tachycardia tendency due to the direct effect of peripheral vasodilator, the inventor has tried designing a chemical compound that has both vasodilatation and β-adrenoceptor blocking agent activation effects. Vanidilol type of β-blocking agent contains a guaiacoxypropanolamines ring, while the previously synthesized vanidipinedilol of this inventor, which belongs to the derivative of Vanidilol, has demonstrated in a series of experiment that it has β-adrenoceptor blocking agent effects.

Furthermore, Asano, M. (J. Pharmacol, 296, pp. 204-211, 1990) has suggested YM-16151-1, and Shibasaki, K. (Gen. Pharmac.29, pp. 545-550, 1997) has demonstrated YM 430. Though both these two compounds have β-adrenoceptor blocking effects as shown in FIG. 1, their structures are different from this invention.

### SUMMARY OF THE INVENTION

Therefore, an object of the invention is to structurally embellish, using Vanidilol as the fundamental key structure. The main purpose is to embellish aldehyde at the 4-position on Vanidilol, and introduce a dihydropyridine ring with vasodilatation effect.

This invention, in part, will also make use of various pharmacological experiments to demonstrate that this 1,4-dihydropiridine derivative chemically with guaiacoxypropanolamine phenoxypropanolamine moiety could continuously maintain hypotension; with activation of competitive β-adrenoreceptor and calcium ion blocking agent; induced vasorelaxing effect; and with activation of calcium ion channel antagonist and β-adrenoreceptor antagonist.

This invention will further demonstrate that by having 1,4-dihydropiridine derivative chemically with guaiacoxypropanolamine phenoxypropanolamine moiety as the main component and adding necessary excipients to form various pharmacological compounds is therapeutically efficient.

The invention, in part, pertains to a compound of formula Ia:

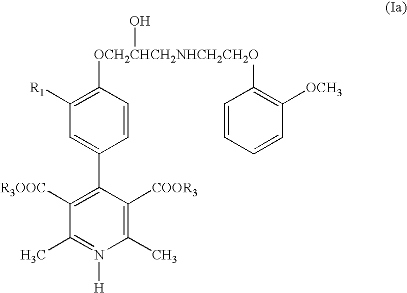

wherein R₁ and R₃ are each individually selected from the group consisting of —X, —H, —NO₂, CF₃, saturated C₁-C₆ alkyl chain, unsaturated C₂-C₆ alkyl chain, saturated C₁-C₆ alkoxy chain, and unsaturated C₂-C₆ alkoxy chain, and wherein X represents a halogen.

The invention, in part, pertains to a pharmaceutical composition that comprises an effective amount of the compound of formula Ia and a pharmaceutically acceptable carrier, diluent or excipient. The invention also pertains to a method for blocking an α-, β-adrenoreceptor or calcium channel that comprises administering to a patient in need thereof an effective amount of the composition containing the compound of formula Ia. Also, a method for inducing hypotension that comprises administering to a patient in need thereof an effective amount of the composition containing the compound of formula Ia. A method for inducing vaso-relaxation comprises administering to a patient in need thereof an effective amount of the composition containing the compound of formula Ia.

The invention, in part, pertains to a compound of formula Ib:

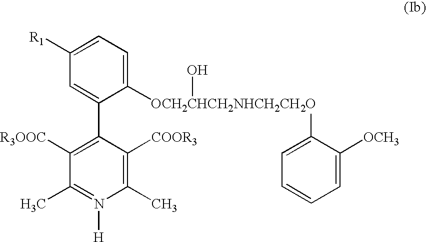

wherein R₁ and R₃ are each individually selected from the group consisting of —X, —H, —NO₂, CF₃, saturated C₁-C₆ alkyl chain, unsaturated C₂-C₆ alkyl chain, saturated C₁-C₆ alkoxy chain, and unsaturated C₂-C₆ alkoxy chain, and wherein X represents a halogen.

The invention, in part, pertains to a pharmaceutical composition that comprises an effective amount of the compound of formula Ib and a pharmaceutically acceptable carrier, diluent or excipient. The invention also pertains to a method for blocking an α-, β-adrenoreceptor or calcium channel that comprises administering to a patient in need thereof an effective amount of the composition containing the compound of formula Ib. Also, a method for inducing hypotension that comprises administering to a patient in need thereof an effective amount of the composition containing the compound of formula Ib. A method for inducing vaso-relaxation comprises administering to a patient in need thereof an effective amount of the composition containing the compound of formula Ib.

### BRIEF DESCRIPTION OF THE DRAWINGS

Table 1 shows the effect of this invention compound on heart rate and hypertensive.

Table 2 shows the inventive compound and its pA₂ value.

Table 3 shows the inventive compound and its pKi value.

FIG. 1 illustrates the structures of compounds YM-16151-1 and YM 430.

FIG. 2 illustrates the synthetic method of this invention compound.

FIG. 3 illustrates the partial structures of this invention compound.

FIG. 4 a)-e) illustrates the inventive compound on heart rate and hypertensive responses:

FIG. 4 a) 0.1 mg/kg of compound 1,

FIG. 4 b) 0.25 mg/kg of compound 1,

FIG. 4 c) 0.5 mg/kg of compound 1,

FIG. 4 d) 1.0 mg/kg of compound 1, and

FIG. 4 e) 2.0 mg/kg of compound 1.

FIG. 5 a)-b) illustrates heart rate and hypertensive responses:

FIG. 5 a) 0.5 mg/kg of compound 1, and

FIG. 5 b) 0.5 mg/kg of nifedipine.

FIG. 6 illustrates a competitive blocking agent L-isoproterenol on increasing heart rate.

| | | | |
|---|---|---|---|
| 1 | control | 2 | 10⁻⁷ M compound 1 |
| 3 | 10⁻⁶ M compound 1 | 4 | 10⁻⁵ M compound 1 |

FIG. 7 illustrates a competitive blocking agent L-isoproterenol on increasing heart rate.

| | | | |
|---|---|---|---|
| 1 | control | 2 | 10⁻⁷ M compound 1 |
| 3 | 10⁻⁶ M compound 1 | 4 | 10⁻⁵ M compound 1 |

FIG. 8 illustrates a competitive blocking agent L-isoproterenol on bronchoconstrictive effect.

| | | | |
|---|---|---|---|
| 1 | control | 2 | 10⁻⁷ M compound 1 |
| 3 | 10⁻⁶ M compound 1 | 4 | 10⁻⁵ M compound 1 |

FIG. 9 illustrates L-isoproterenol on right atrial rate.

| | | | |
|---|---|---|---|
| 1 | L-isoproterenol | 2 | propranolol |
| 3 | Vanidilol | 4 | compound 1 |

FIG. 10 illustrates L-isoproterenol on left atrial systole tension.

| | | | |
|---|---|---|---|
| 1 | L-isoproterenol | 2 | propranolol |
| 3 | Vanidilol | 4 | compound 1 |

FIG. 11 illustrates the effect of this invention compound on tracheal relaxant.

| | | | |
|---|---|---|---|
| 1 | L-isoproterenol | 2 | propranolol |
| 3 | Vanidilol | 4 | compound 1 |

FIG. 12 illustrates the effect of this invention compound on tracheal relaxant.

| | | | |
|---|---|---|---|
| 1 | control | 2 | 10⁻¹⁰ M ICI 118, 551 |
| 3 | 10⁻⁹ M ICI 118, 551 | 4 | 10⁻⁸ M ICI 118, 551 |

FIG. 13 illustrates the effect of this invention compound on tracheal relaxant.

| | | | |
|---|---|---|---|
| 1 | control | 2 | 10⁻⁸ M ICI 118, 551 |

FIG. 14 a)-b) illustrates this invention compound on receptor binding:

FIG. 14 a) [³H]CGP-12177 bound, and

FIG. 14 b) protein bound

FIG. 15 illustrates competitive curve of β-adrenoceptor blocking agent:

| | | | |
|---|---|---|---|
| 1 | compound 1 | 2 | propranolol |

FIG. 16 a)-b) illustrates this invention compound on combined receptors:

FIG. 16 a) [³H]CGP-12177 bound, and

FIG. 16 b) protein bound

FIG. 17 illustrates competitive curve of β-adrenoceptor blocking agent

| | | | |
|---|---|---|---|
| 1 | compound 1 | 2 | propranolol |

FIG. 18 illustrates the effect of this invention compound on atrial rate.

| | | | |
|---|---|---|---|
| 1 | control | 2 | 10⁻⁷ M compound 1 |
| 3 | 10⁻⁶ M compound 1 | 4 | 10⁻⁵ M compound 1 |

FIG. 19 illustrates the effect of this invention compound on atrial rate.

| | | | |
|---|---|---|---|
| 1 | control | 2 | 10⁻⁷ M compound 1 |
| 3 | 10⁻⁶ M compound 1 | 4 | 10⁻⁵ M compound 1 |

FIG. 20 illustrates the effect of this invention compound on vasorelaxant.

| | | | |
|---|---|---|---|
| 1 | control | 2 | 10⁻⁸ M compound 1 |
| 3 | 10⁻⁷ M compound 1 | 4 | 10⁻⁶ M compound 1 |
| 5 | 10⁻⁵ M compound 1 | | |

FIG. 21 illustrates pre-treatment with Bay K 8644 will affect vasorelaxant effects.

| | | | |
|---|---|---|---|
| 1 | control | 2 | 10⁻⁸ M compound 1 |
| 3 | 10⁻⁷ M compound 1 | 4 | 10⁻⁶ M compound 1 |
| 5 | 10⁻⁵ M compound 1 | | |

FIG. 22 illustrates pre-treatment with Bay K 8644 will affect vasorelaxant effects.

| | | | |
|---|---|---|---|
| 1 | control | 2 | Treatment before Bay K 8644 |

FIG. 23 a)-b) illustrates [³H]nitrendipine on receptor binding:

FIG. 23 a) [³H]nitrendipine receptor bound, and

FIG. 23 b) protein bound.

FIG. 24 illustrates competitive curve of calcium ion entry blocking agent.

| | | | |
|---|---|---|---|
| 1 | nifedipine | 2 | compound 1 |

FIG. 25 illustrates fluorescence detection of calcium ion.

| | | | |
|---|---|---|---|
| 1 | 50 mM potassium chloride | 2 | 10⁻⁴ M A23187 |
| 3 | 5 mM EDTA | | |

FIG. 26 illustrates fluorescence detection of calcium ion.

| | | | |
|---|---|---|---|
| 1 | 10⁻⁶ M compound 1 | 2 | 50 mM KCl |
| 3 | 10⁻⁴ M A23187 | 4 | 5 mM EDTA |

FIG. 27 illustrates fluorescence detection of calcium ion.

| | | | |
|---|---|---|---|
| 1 | control | 2 | 50 mM potassium chloride |
| 3 | 10⁻⁸ M compound 1 + 50 mM | | |
| | potassium chloride | | |
| 4 | 10⁻⁷ M compound 1 + 50 mM | | |
| | potassium chloride | | |
| 5 | 10⁻⁶ M compound 1 + 50 mM | | |
| | potassium chloride | | |

FIG. 28 illustrates fluorescence detection of calcium ion.
1 . . . 10 μM Bay K8644

FIG. 29 illustrates fluorescence detection of calcium ion.

| | | | |
|---|---|---|---|
| 1 | 10⁻⁶ M compound 1 | 2 | 10 μM Bay K 8644 |
| 3 | 10⁻⁴ M A23187 | 4 | 5 mM EDTA |

FIG. 30 illustrates fluorescence detection of calcium ion.

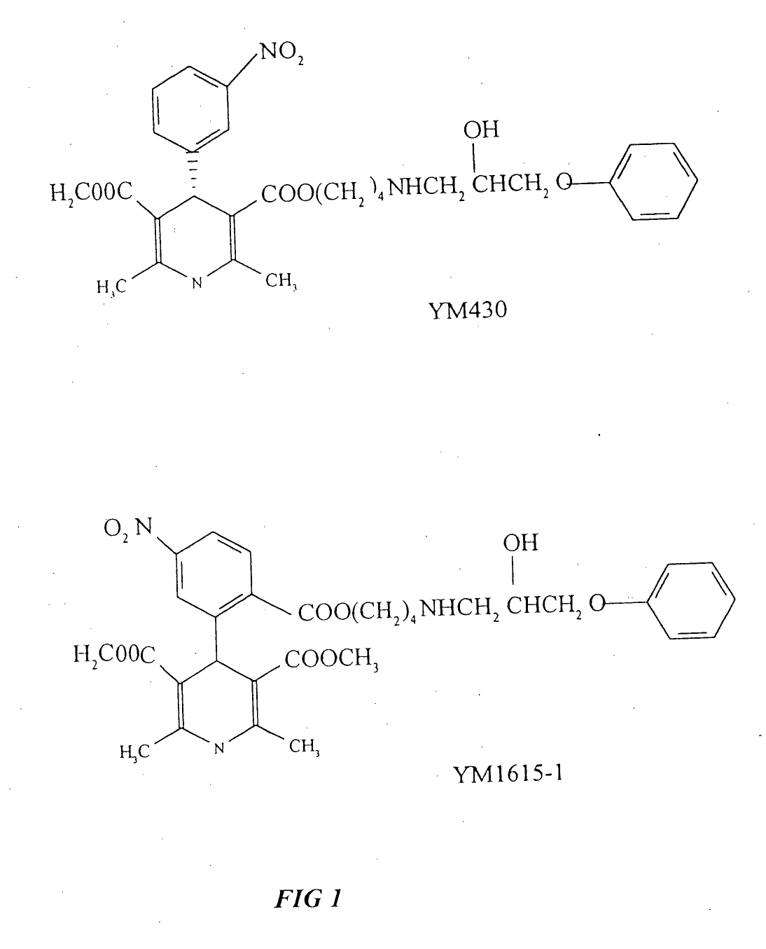

| | |
|---|---|
| 1 | control |
| 2 | 10 μM Bay K 8644 |
| 3 | 10⁻⁸ M compound 1 + 10 μM Bay K 8644 |
| 4 | 10⁻⁷ M compound 1 + 10 μM Bay K 8644 |
| 5 | 10⁻⁶ M compound 1 + 10 μM Bay K 8644 |

### DETAILED DESCRIPTION

The invention discloses some 1,4-dihydropiridine derivative compounds bonded chemically with guaiacoxypropanolamine and phenoxypropanolamine moiety.

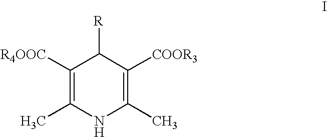

The compounds of 1,4-dihydropiridine derivative has the formula I, wherein R is selected from the four groups as follow:

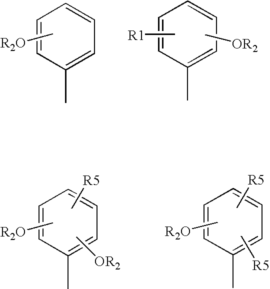

R₁ is selected from H, X, NO₂, saturated C₁-C₆ alkyl group, unsaturated C₁-C₆ alkyl group, R₂ selected from H, CH₃, and the group of

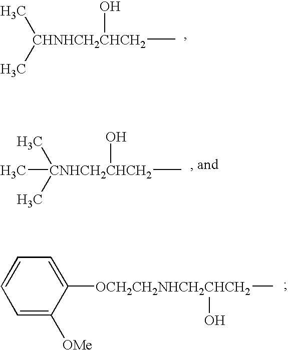

R₃ and R₄ are individually selected from saturated C₁-C₆ alkyl group, and unsaturated C₁-C₆ alkyl group; R₅ selected from OH, saturated C₁-C₆alkyl group, and unsaturated C₁-C₆ alkyl group.

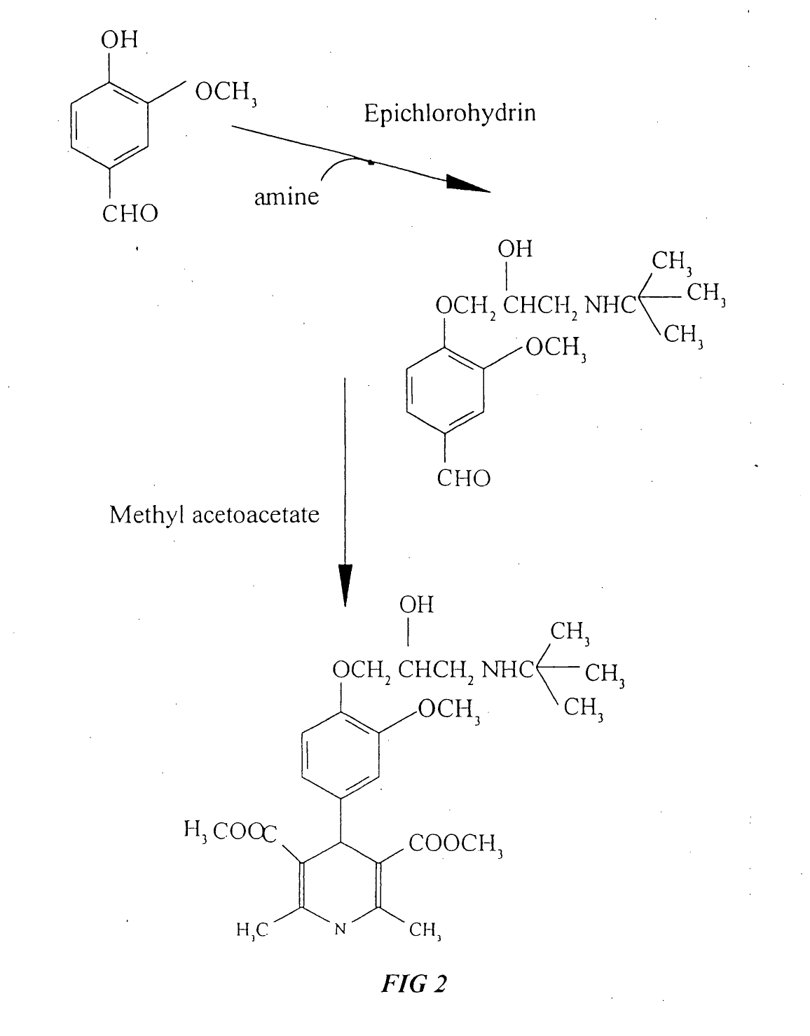

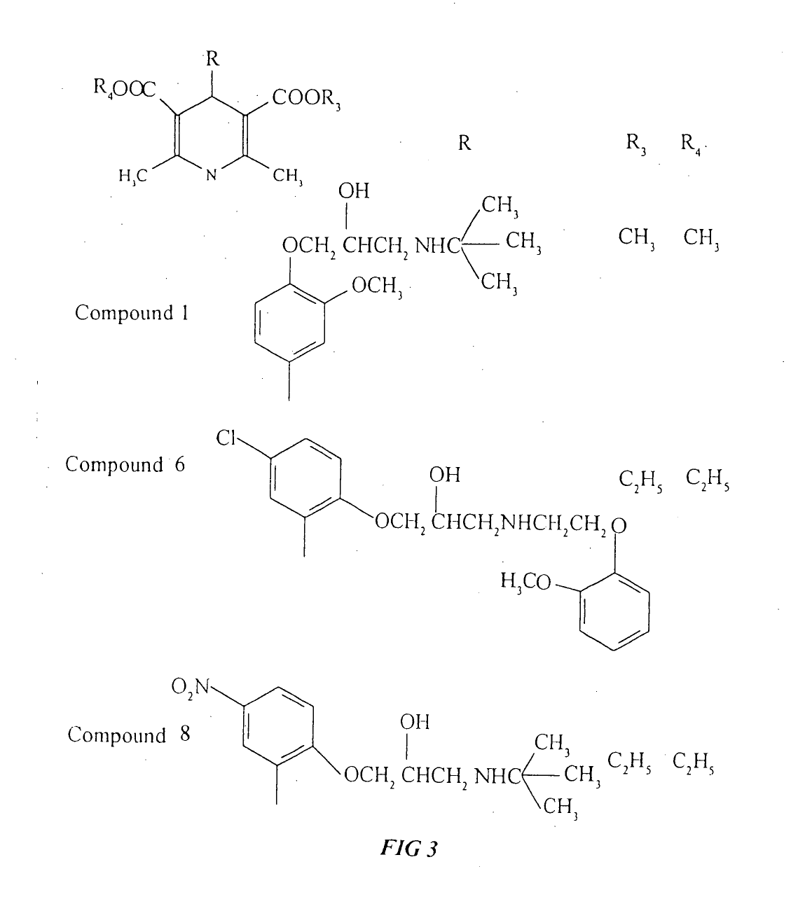

The 1,4-dihydropiridine derivative bonds chemically with guaiacoxypropanolamine based phenoxypropanolamine moiety, which has formula I as the main structure, adopts the method for compound synthesis as shown in FIG. 2, which could be roughly differentiated into 4 types.

A preferred embodiment of the invention includes a compound of the formula Ia:

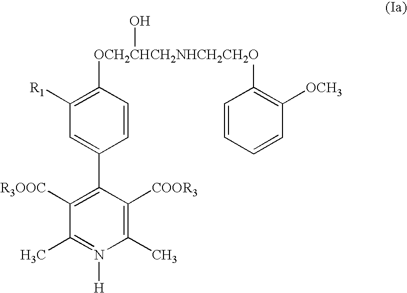

wherein R₁ and R₃ are each individually selected from the group consisting of —X, —H, —NO₂, CF₃, saturated C₁-C₆ alkyl chain, unsaturated C₂-C₆ alkyl chain, saturated C₁-C₆ alkoxy chain, and unsaturated C₂-C₆ alkoxy chain, and wherein X represents a halogen.

The preferred embodiments include a pharmaceutical composition that comprises an effective amount of the compound of formula Ia and a pharmaceutically acceptable carrier, diluent or excipient. The invention also pertains to a method for blocking an α-, β-adrenoreceptor or calcium channel that comprises administering to a patient in need thereof an effective amount of the composition containing the compound of formula Ia. Also, a method for inducing hypotension that comprises administering to a patient in need thereof an effective amount of the composition containing the compound of formula Ia. A method for inducing vaso-relaxation comprises administering to a patient in need thereof an effective amount of the composition containing the compound of formula Ia.

A preferred embodiment of the invention includes a compound of the formula Ib:

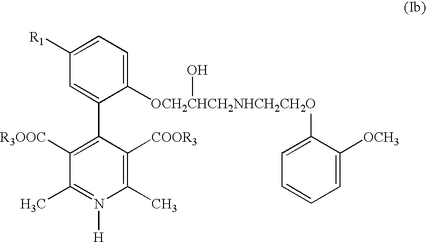

wherein R₁ and R₃ are each individually selected from the group consisting of —X, —H, —NO₂, CF₃, saturated C₁-C₆ alkyl chain, unsaturated C₂-C₆ alkyl chain, saturated C₁-C₆ alkoxy chain, and unsaturated C₂-C₆ alkoxy chain, and wherein X represents a halogen.

The preferred embodiments include a pharmaceutical composition that comprises an effective amount of the compound of formula Ib and a pharmaceutically acceptable carrier, diluent or excipient. The invention also pertains to a method for blocking an α-, β-adrenoreceptor or calcium channel that comprises administering to a patient in need thereof an effective amount of the composition containing the compound of formula Ib. Also, a method for inducing hypotension that comprises administering to a patient in need thereof an effective amount of the composition containing the compound of formula Ib. A method for inducing vaso-relaxation comprises administering to a patient in need thereof an effective amount of the composition containing the compound of formula Ib.

Preparation Method of 1,4-Dihydropiridine Derivative Compounds

Method 1

4-hydroxy-3-methoxy-1-benzaldehyde was dissolved in ethanol with sodium hydroxide solution. After reacted with epichlorohydrin, decompressed to condense and crystallized, N-[4-(2,3-epoxy-propoxy)-3-methoxy]-1-benzaldehyde was obtained. Then with tert-butylamine to undergo amination, N-{4-[2-hydroxy-3-(tert-butylamino)propoxy]-3-methoxy}-1-benzaldehyde was obtained. By adding methylacetoacetate, Ethanol, and concentrated NH₃ solution to N-{4-[2-hydroxy-3-(tert-butylamino)propoxy]-3-methoxy}-1-benzaldehyde, well mixed then heat under reflux; directly decompressed to condense and Ethanol removed, the remaining solution was mixed with saturated NaCO₃ solution, and extracted with CHCl₃ and water. Then it was purified by column chromatography, and repeatedly re-crystallized the solid to obtain compound 1.

Say the amination, if 2-methoxy-1-oxyethylamino benzene is used, with similar procedure as the above mentioned, purified compound 2 will be obtained. On the other hand, if the material methylacetoacetate is replaced with ethylacetoacetate and the procedure remained as the above mentioned, purified compound 3 will be obtained.

Method 2

2-chloro-4-hydroxy-benzaldehyde was dissolved in ethanol with sodium hydroxide solution. After epichlorohydrin reacted; decompressed to condense and crystallized, N-[4-(2,3-epoxy-propoxy)-2-chloro]-1-benzaldehyde was obtained. Then with N-{4-2-methoxy-1-oxyethylaminobenzene to undergo amination, N-{4-[2-hydroxy-3-(2-methoxy-1-oxyethylamino-benzene)propoxy]-2-chloro}-1-benzaldehyde was obtained.

By adding ethylacetoacetate, Ethanol, and concentrated NH₃ solution to N-{4-[2-hydroxy-3-(2-methoxy-1-oxyethylamino-benzene)propoxy]-2-chloro}-1-benzaldehyde; well mixed then heat under reflux; directly decompressed to condense and Ethanol removed, the remaining solution was mixed with saturated NaCO₃ solution and extracted with CHCl₃ and water. Then it was purified by column chromato Figure, and re-crystallized the solid to obtain compound 7.

Method 3

When 5-Chlorosalicylaldehyde was dissolved in ethanol with sodium hydroxide solution, and undergone epichlorohydrin reaction; decompressed to condense and crystallized, N-[2,3-epoxypropoxy]-5-chloro]-1-benzaldehyde was obtained. Then with tert-butylamine to undergo amination, N-{2-[2-hydroxy-3-(tert-butylamino)propoxy]-5-chloro}-1-benzaldehyde was obtained. By adding ethylacetoacetate, Ethanol, and concentrated NH₃ solution to N-{2-[2-hydroxy-3-(tert-butylamino)propoxy]-5-chloro}-1-benzaldehyde; well mixed then under reflux; directly decompressed to condense and Ethanol removed, the remaining solution was mixed with saturated NaCO₃ solution and extracted with CHCl₃ and water. Then it was purified by column chromatography, and re-crystallized to obtain compound 4.

When amination, if n-butylamine and 2-methoxy-1-oxyethylaminobenzene is used separately, with similar procedure as the above mentioned, purified compound 5 and compound 6 will be obtained.

Method 4

When 5-nitrosalicylaldehyde was dissolved in ethanol with sodium hydroxide solution, and undergone epichlorohydrin reaction; decompressed to condense and crystallized, N-[2-(2,3-epoxypropoxy]-5-nitro]-1-benzaldehyde was obtained. Then with tert-butylamine to undergo amination, N-{2-[2-hydroxy-3-(n-butylamino)propoxy]-5-chloro}-1-benzaldehyde was obtained. By adding ethylacetoacetate, Ethanol, and concentrated NH₃ solution to N-{2-[2-hydroxy-3-(tert-butylamino)propoxy]-5-chloro}-1-benzaldehyde; well mixed then reflux; directly decompressed to condense and Ethanol removed, the remaining solution was mixed with saturated NaCO₃ solution and extracted with CHCl₃ and water. Then it was purified by column chromato Figure, and re-crystallized to obtain compound 8.

When amination, if 2-methoxy-1-oxyethylaminobenzene is used, with similar procedure as the above mentioned, purified compound 9 will be obtained.

2-methoxy-1-oxyethylaminobenzene could be prepared by adding sodium hydroxide to warm mixture of 2-methoxyphenol and ethylene dibromide. After continuous reflux; decompressed to condense and crystallized, 2-methoxy-1-oxyethylbromide benzene is obtained.

2-methoxy-1-oxyethylbromide benzene and potassium phthalimide were mixed, and the mixture dissolved in dimethylformamide. With continuous reacted the mixture and stay overnight, 2-methoxy-1-oxyethylphthalimide benzene was obtained after re-crystallization. Warming 2-methoxy-1-oxyethyl phthalimide benzene and hydrazine hydrate will result in 2-methoxy-1-oxyethylaminobenzene.

After purification and crystallization, the products are individually tested for their physio-chemical information, including element analysis; MS, IR, ¹H-NMR (CDCl₃), and UV etc. Appropriate experimental models are used to evaluate their pharmacological activities, thus ascertain the compound's activity.

The compound of this invention will include various excipients; carriers or diluents and pharmaceutically approved pH of processed salts in accordance to necessity to form composition with therapeutic efficacy. Such pharmaceutical preparation could be in solid form for oral and rectum administration; liquid form or non-intestinal injection form; or ointment form for direct application on affected part. Such solid forms are manufactured according to common pharmaceutical preparation methods, which will include disintegrant like starch; sodium carboxymethyl cellulose, adhesive like ethanol; glycerine, or magnesium stearic acid; lactose to make into pharmaceutical preparation like tablets or filled into capsules or suppository. Solution or saline that include this invention compound as ingredient could use buffers of phosphoric nature to adjust the pH to suitable level, before adding adjutant; emulsifier to produce injection dose or other liquid preparation. This invention compound or pharmaceutical manufacturing could mixed synthetic acid salts with various fundamental preparations to form ointments according to known pharmaceutical manufacturing methods. Pharmaceutical compounds manufactured with this invention compound being the major ingredient could be used on mammals to produce the efficacy of this main ingredient. General dosage could be adjusted according to the degree of symptoms, and normally a person will require 50 to 300 mg each time, three times per day.

Pharmaceutical Activity

This inventive compound has been proven by the following pharmaceutical experiments that it has β-receptor and calcium ion entry blocking agent; selectivity towards integrated β-adrenoceptor; and whether has intrinsic sympathomimetic activity, ISA; such type of compounds has tracheal dilation and vasorelaxant effects. Laser cell detector (ACAS 570) was used to determine the changes in the cell calcium ion concentration of smooth muscle cell line A7r5.

Heart Rate and Blood Pressure in Living Rat

Male Wistar rats, weighing 200˜300 g were abdominal anaesthetized with pentobarbital sodium. Tracheal was cannulated to maintain normal respiration. Polyethylene tube was inserted into the left femoral vein to facilitate drug administration. A 3-way stopcock was used, with one end connected to a syringe for drug injection, while the other end was connected to the syringe filled with physiological saline. The latter was used to prevent residual drugs in the polyethylene tube after injection, which would affect experimental accuracy.

The right femoral artery was also inserted with polyethylene, and a 3-way stopcock was used too, where one end was connected to heparin solution to prevent embolism. The other end was connected to a Disposable Diaphragm Dome, TA1019, and linked to a transducer. Through an amplifier, a recorder recorded the overall and average arterial pressure; heart rate to evaluate the effect of drug on blood pressure and heart rate. Different concentrations 0.1; 0.25; 0.5; 1.0; 2.0 mg/kg of compound 1 were given to the rats via femoral vein and the differences in the heart rate and blood pressure were compared. Furthermore in another group, compound 2, 3, 4, 5, 6, 7, 8, and 9 of concentration 1.0 mg/kg were separately given to different rats via femoral vein, and the differences in the heart rate and blood pressure were compared.

Results

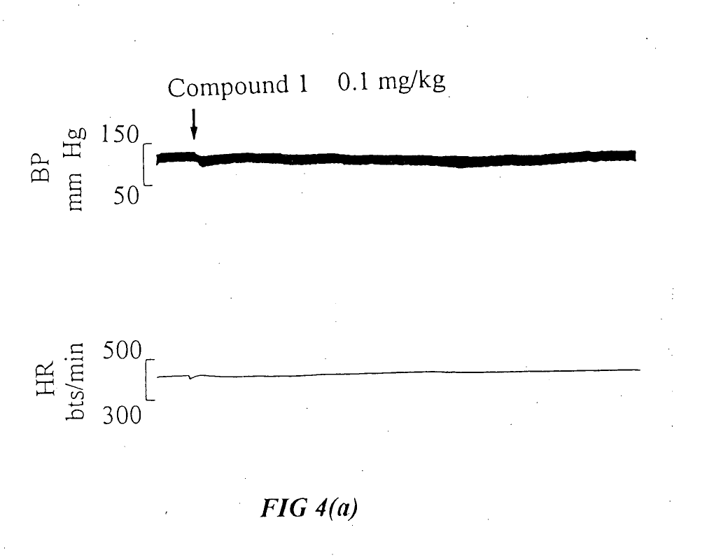

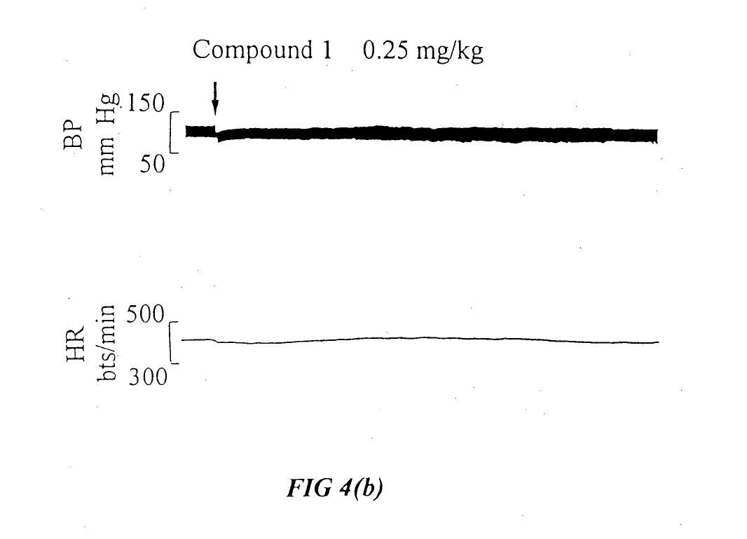

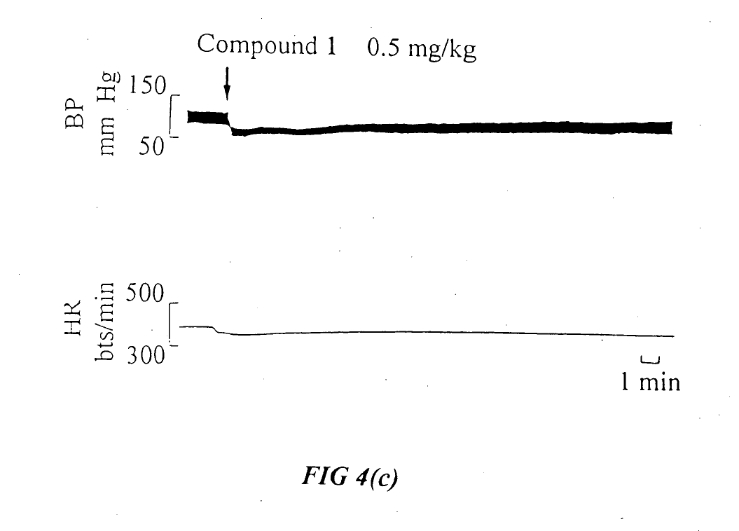

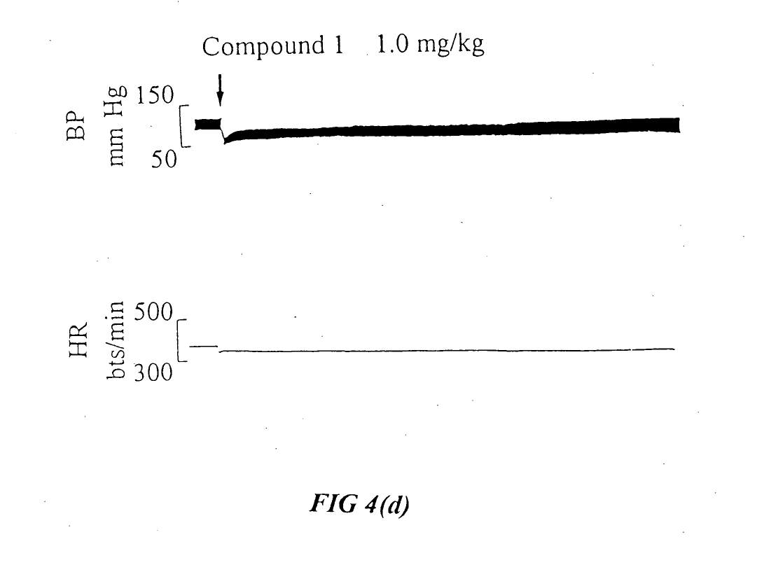

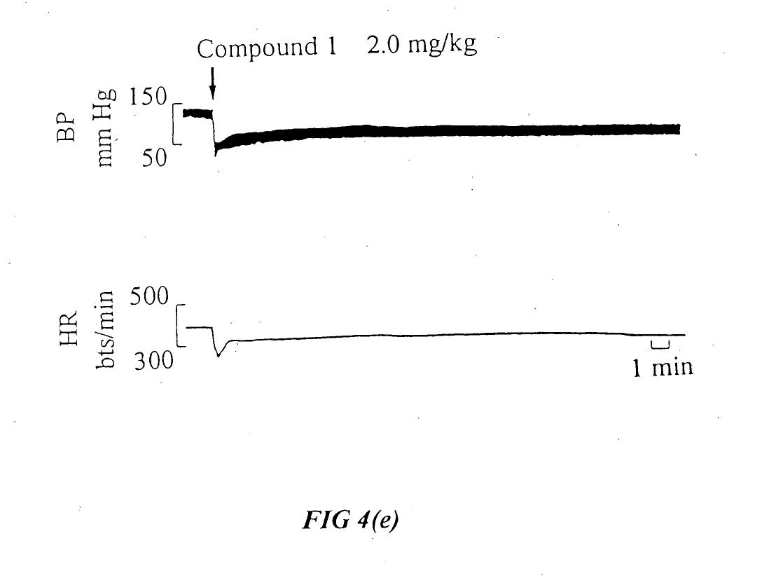

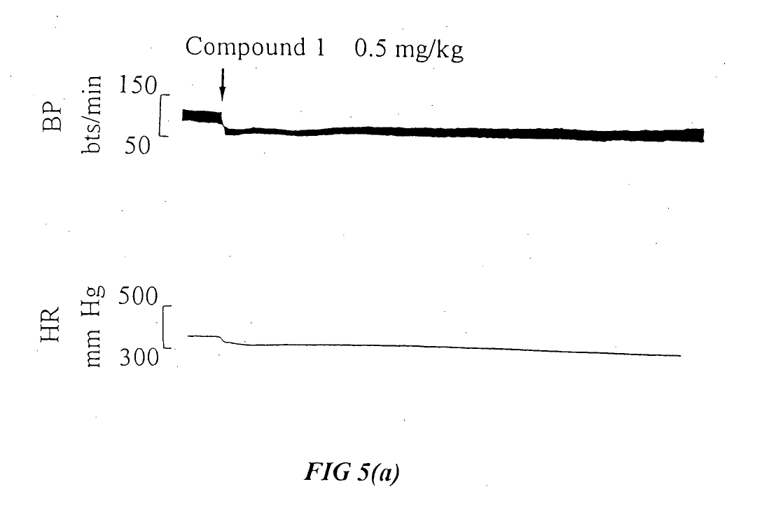

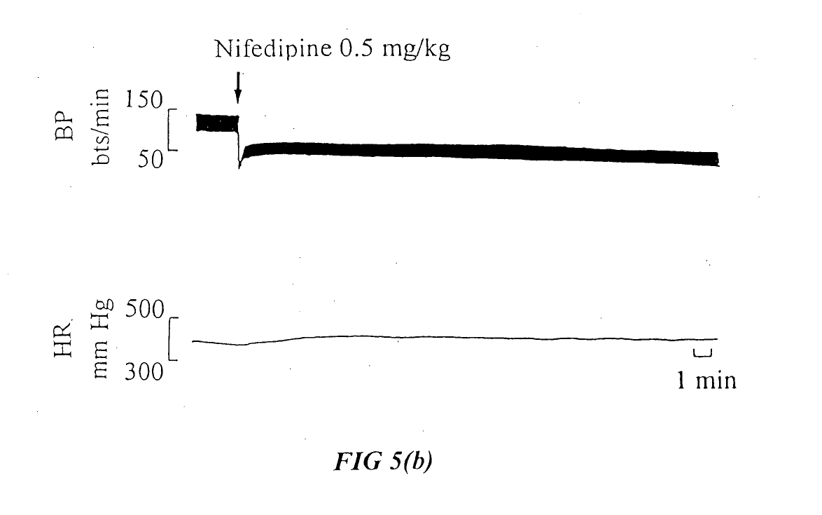

Intravenous injection of different concentration of compound 1 produced a continuous dose-dependent blood pressure lowering effect without increasing heart rate for approximately 1 hour in normotensive rats (FIGS. 4(a)˜(e)). In this experiment, intravenous injection of 0.5 mg/kg of nifedipine produced a significant blood pressure lowering effect, accompanied by reflex tarchycardia (FIGS. 5(b)). However, such observation was not found in compound 1 (FIGS. 5(a)), instead, a decrease in heart rate was shown, thus could prevent reflex tarchycardia side effect produced by calcium ion entry blocking agent of 1,4-dihydropiridine nature. The effects of compound 1-9 on heart rate and blood pressure were shown in Table 1.

Atrium; Tracheal Experiments on Isolated Rat Tissues

The methods published by Chen, I. J et. al. in Gen Pharmacol. 24, pp. 1425-1433 (1993) and by Sheu, M. M. et. al. in Pharmacology 54, pp. 211-224 (1997) were referenced and modified.

The entire heart of rat was removed immediately after the rat was sacrificed and the blood drained by incised carotid arteries, and placed in Kreb-Henseleit solution equilibrated to a mixture of 95% O₂ and 5% CO₂ at room temperature (20˜25° C.). The right and left atria were then separated. The spontaneously-beating right atria was clipped on both end by heart shaped clips, where one end was fixed at the bottom of 10 ml of tissue bath made of physiological saline solution, and temperature maintained at 37° C. The other end of the atria was connected to a force transducer, and isometric contractions and beating rate of the right atria were recorded by COULBOURN AT-High-Speed VideoFigure. After the samples were given 250 mg of contractions and reach equilibrium the following experiments were carried out:
(a) β-adrenoceptor blocking action

When the spontaneously beating rate of right atria reached a certain stability, cumulative administration of L-isoproterenol from 1×10⁻¹⁰˜3×10⁻¹⁰ M caused the heart rate to increase continuously, and a cumulative dose-response curve was obtained. Then the L-isoproterenol was thoroughly washed off with Kreb's solution to recover the right atria's heart rate stability. After the equilibrium was reached again for at least 60 minutes, different concentrations (10⁻⁷, 10⁻⁶, 10⁻⁷ M) of compound 1 were added. 30 minutes later, cumulative administration of L-isoproterenol from 1×10⁻¹⁰˜3×10⁻¹⁰ M were carried out again, and another new cumulative dose-response curve was obtained. Administration of L-isoproterenol started from concentration 1×10⁻¹⁰ M, and the concentration was raised 0.5 log each time for a total of six times. Cumulative administration interval was when the previous concentration reached its greatest effect, the next concentration would be immediately given. The time interval was approximately 3˜5 minutes, and the EC₅₀ value could be obtained. From Schild plots, the pA₂ of compound 1 could be found. In other groups of rats, after separate administration of compound 2, 3, 4, 5, 6, 7, 8, and 9, their pA₂ values were obtained.

(b) Calculation of pA₂ value:

According to the method mentioned by Arunlakshana, O. et al in Br. J. Pharmacol. 14, pp. 48-57 (1959), which used the logarithm values of compound concentration testings as the x-coordinates, and the logarithm values of blocking agent of similar effect and (dose ratio)⁻¹ as the y-coordinates, the data obtained were plotted into Figures and the slope of regression found. From x-coordinates of the line of regression, the intercept value was found, which is the pA₂ value of the compound under testing. The equation is as follows:
pA₂=−Log KB Log(DRADJ−1)=nlog[B]−LogKB

$$DRADJ ( dose ratio adjusted ) = \frac{DR ( dose ratio )}{CF ( correction factor )}$$

[B] Test compound concentration in moles
KB: equilibrium dissociation constant
n: value of slope DR: test EC₅₀ divided by control EC₅₀
CF: EC₅₀ of second or third control groups divided by EC50 of first control group
   (c) Effect of Compound on the Increase of Spontaneous Beating in Right Atrium Caused by CaCl₂

The right atrium of the rats was allowed to equilibrate in Kreb's solution for at least 60 minutes. When the spontaneous beating rate had reached a certain stability, CaCl₂ of different concentrations (3.0, 6.0, and 9.0 mM) were cumulatively administered, and the changes in the spontaneous beating of right atrium were observed. Then the right atrium was thoroughly washed with Kreb's solution for several times and re-equilibrated for at least 60 minutes before different concentrations (10⁻⁷, 10⁻⁶, and 10⁻⁵ M) of compound 1 were administered. 30 minutes later, different concentrations (3.0, 6.0, and 9.0 mM) were cumulatively administered, and the changes in the spontaneous beating of right atrium were observed again. The effect of CaCl₂ on the changes of spontaneous beating in right atrium were compared with and without the presence of compound 1.

(2) Experiments on the Isolated Left Atrial Tissue of Rat

The inspontaneously-beating left atrial tissue was obtained from rat's isolated right atrial tissue experiments. Under similar conditions, contractions were induced in the right atria by approximately 1 volt of square waves which had a wave width about 1 msec wider than the threshold voltage. The contraction rate was 1 Hz and the resting tension 0.5 gm. After 60 minutes of equilibration, the following experiments were performed:

(a) Completion of cumulative concentration-response curve: similar to experimental method on isolated right atrium;

(b) Calculation of pA2 value: similar to calculation method on isolated right atrium.

RESULTS

Effect of Compound 1 on β-Adrenoreceptor (β₁) Activity:

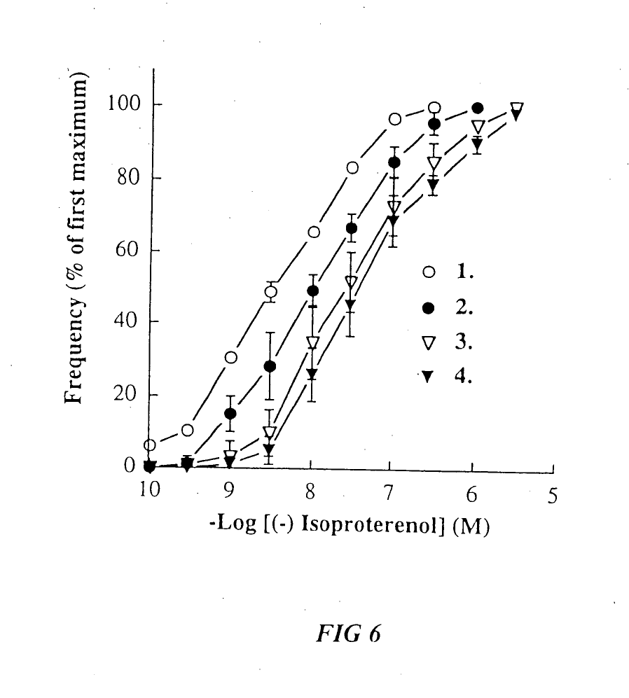

Using the spontaneously beating function of Wistar rat's isolated right atrium and cumulatively administered different dosages (1×10⁻¹⁰˜1×10⁻⁶ M) of L-isoproterenol resulted in continuous increment of heart rate, where a cumulative dose-response curve was obtained. As shown in FIG. 6, different concentrations (10⁻⁶, 10⁻⁵, and 10⁻⁶ M) of compound 1 could competitively block the heart rate increment effect of L-isoproterenol, at the same time, the cumulative concentration-response curve of L-isoproterenol indicated a dose-dependant movement from left to right.

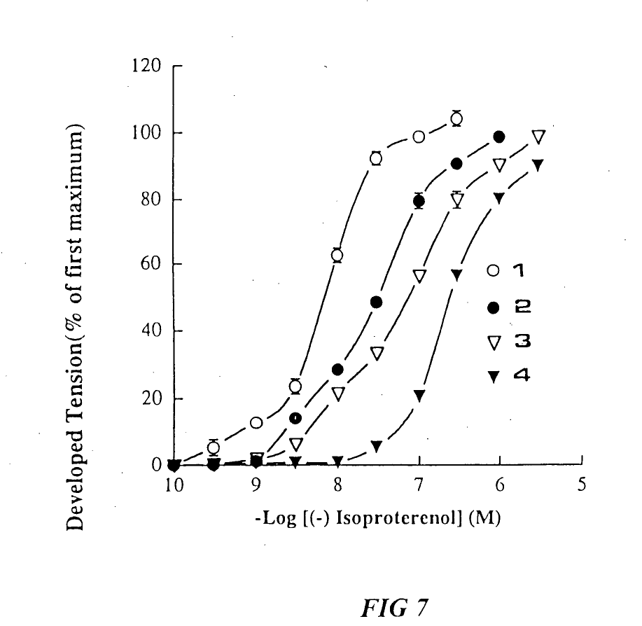

Furthermore, by electrically excite the Wistar rat's isolated left atrium before cumulative administered L-isoproterenol could increase contractility. Similarly, as shown in FIG. 7, different concentrations (10⁻⁶, 10⁻⁵, and 10⁻⁶ M) of compound 1 could competitively block the heart contractility increment effect of L-isoproterenol, at the same time, the cumulative concentration-response curve of L-isoproterenol indicated a dose-dependant movement from left to right.

The pA₂ value of compound 1 in Wistar rat's isolated right atrium experiment was 7.21±0.32; and the pA₂ value left atrium contractility experiment was 6.91±0.26. The detailed values of pA₂ and rate of regression slope of the other compounds were indicated in Table 2.

(3) Experiments on Guinea Pig's Isolated Tracheal

Guinea pigs of weight between 300˜500 gm were used. 18˜24 hours before the experiment, 5 mg/kg of reserpine was injected via abdominal cavity to prevent the discharge of catecholamines, as suggested by O'Donnell and Wanstall (1979), due to the administration of phenoxybenzamine during the experimental process. After the guinea pigs were sacrificed, a slit was made along the neck, and a portion of tracheal approximately 4 cm long was removed. The tracheal was then placed in Kreb's solution aerated with a mixture of 95% O₂ and 5% CO₂ and maintained at room temperature. After the surrounding tissue was carefully removed, the tracheal was cut into spiral shape with every turn having 3˜4 cartilage segments, and divided according to the method suggested by Constantine (1965). The two ends of the tracheal were clamped with frog-heart shaped clamps, one end was fixed at the bottom of tissue bath filled with 20 ml of Kreb's solution, maintained at 37° C., while the other end was connected to a force transducer. Through a COULBOURN AT-High-Speed Videograph, long isometric contractions were recorded. After the sample was given 1.5 gm of tension and equilibrated, the following experiments were performed:

(a) Cumulative concentration response curve:

In the experiment, tracheal was first treated with 50 μm phenoxybenzamine for 30 minutes to prevent extraneuronal uptake, and reduce L-isoproterenol effect suggested by O'Donnell and Wanstall (1976). Then the tracheal was repeatedly washed with Kreb's solution for 20 minutes and 10−6 M of carbochol was added to cause contraction in the guinea pig's tracheal. When the contraction reached the maximum, every division of tracheal was used to complete two concentration response curve of L-isoproternol, one of them without administration of test compound and used as control; while the other curve was administered with compound 1 for 30 minutes before concentration response curve was completed. This is the test group.

(b) Calculation of pA₂: similar to the calculation method for isolated right atrium experiment.

Results

Effect of Compound on β-Adrenoreceptor (β₁) Activity:

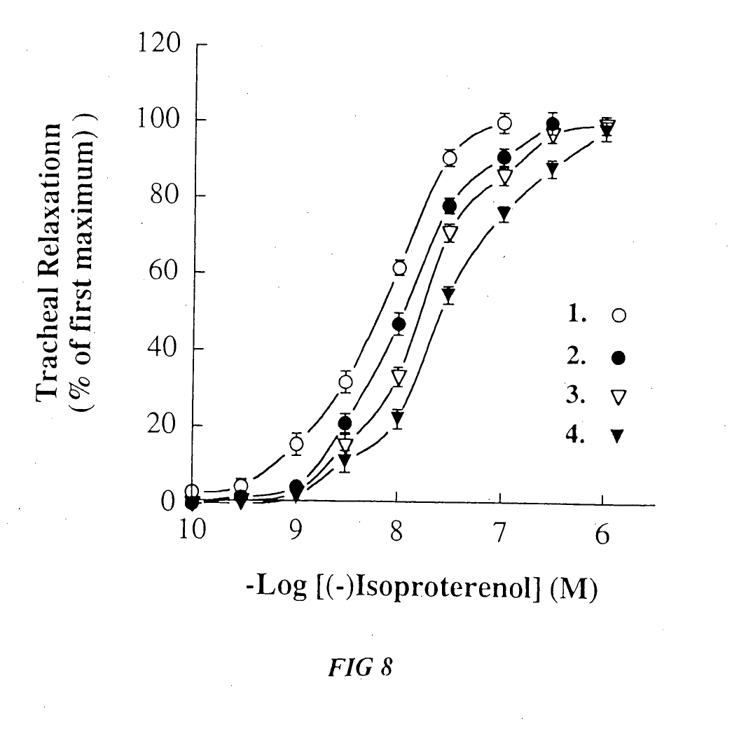

10⁻⁶ M of carbachol was used to cause contraction in guinea pig's isolated tracheal. When it reached stability, cumulative administration of L-isoproterenol was used to obtain tracheal tension-relaxation curve. As shown in FIG. 8, treatment with 10⁻⁶, 10⁻⁵, and 10⁻⁶ M of compound 1 prior experiment could competitively block the effect of L-isoproterenol. The cumulative concentration-response curve of L-isoproterenol blocking indicated a dose-dependant movement from left to right. The pA₂ value of compound 1 in guinea pig's isolated tracheal experiment was 7.09±0.54. The detailed values of pA₂ and rate of regression slope of the other compounds were indicated in Table 2.

(4) Discussion on the Direct Effect of Wistar Rat's Isolated Atria:

With reference to the method suggested by Kaumann, A. J. et. al. in Naunyn-Schmiedeberg's Arch. Pharmacol 311, pp. 205-218 and pp 237-248 (1980). Wistar rats of weight between 200˜300 gm were used. 18˜24 hours before the experiment, 5 mg/kg of reserpine was injected via abdominal cavity to remove all endogenous catecholamines. Prior experiment, Wistar rats were sacrificed, the heart was immediately removed and placed in Kreb's solution aerated with air mixture and maintained at room temperature. The right and left atria were carefully separated. Then in accordance to the above-mentioned experimental method, the effects of cumulative administration of compound 1 (10⁻¹⁰ M˜3×10⁻⁶ M) on right atrium were recorded.

Results

The Selectivity of Compound 1 on β₁:β₂ Types of β-Adrenoreceptors

β₁ type: The selectivity ratio of β₁ type adrenoceptor was obtained from the negative logarithm of the average pA₂ difference between right atrium and tracheal. This was in accordance to the method published by Baird, J. R. C. et. al. in J. Pharm. Pharmacol 24 pp. 880-885 (1972). The effect of compound 1 on right atrium was 1.32× of tracheal; the effect of Vanidilol on right atrium was 0.98× of tracheal; while the effect of propranolol on right atrium was 1.7× stronger than tracheal. This indicates that compound 1 is similar to Vanidilol and propranolol in that they are non-selective on the type of β-adrenoceptor.

(5) Discussion on the Direct Effect of Guinea Pig's Isolated Tracheal:

With reference to the method suggested by Kaumann, A. J. et. al. in Naunyn-Schmiedeberg's Arch. Pharmacol 311, pp. 205-218 and pp.237-248 (1980). guinea pigs of weight between 350˜500 gm were used. 18˜24 hours before the experiment, 5 mg/kg of reserpine was injected via abdominal cavity to remove all endogenous catecholamines. Prior experiment, guinea pigs were sacrificed, the tracheal was immediately removed and placed in Kreb's solution aerated with air mixture and maintained at room temperature of 22˜25° C. The evaluation of the tracheal's endogenous activity was performed with modification according to the method suggested by Tesfamariam, B. et. al. in Br. J. Pharmacol 112, pp. 55-58 (1994). Firstly, compound 1 of concentrations 10⁻⁷; 10⁻⁶; 10⁻⁵M were cumulatively added into the tissue trough to observe their effects on tracheal. Then the tissue was repeatedly washed with Kreb's solution. After the tissue had been re-equilibrated for 60 minutes, ICI 118, 551 were administered. After treatment for 30 minutes, compound 1 of similar concentration was administered, and the intrinsic sympathomimetic activity of tracheal was observed.

Results

The Intrinsic Sympathomimetic Activity of Compound 1

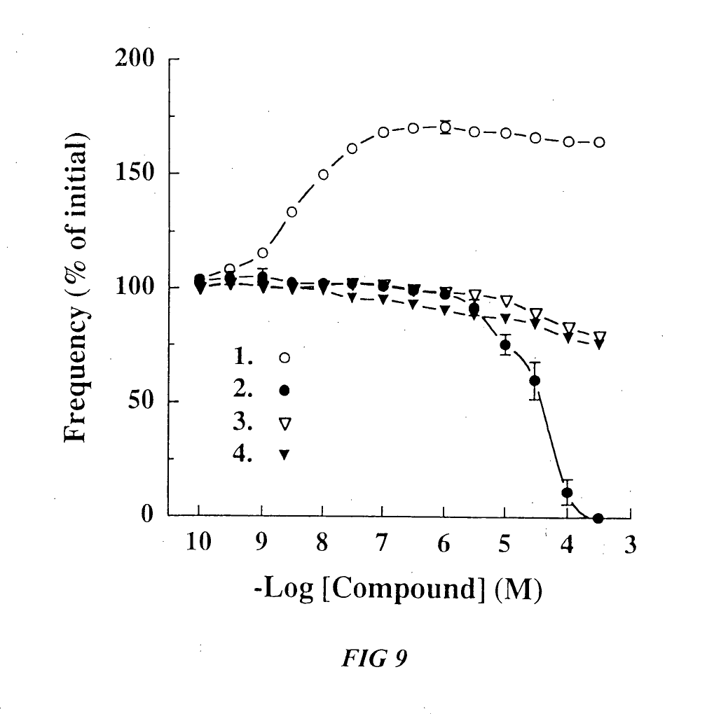

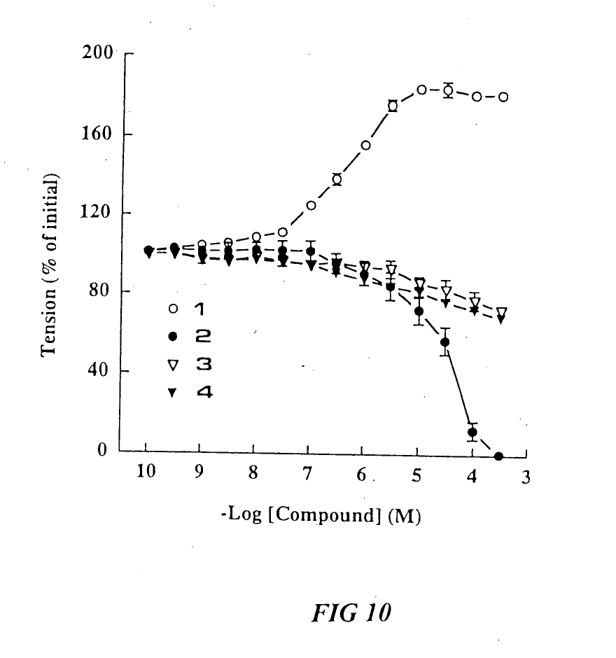

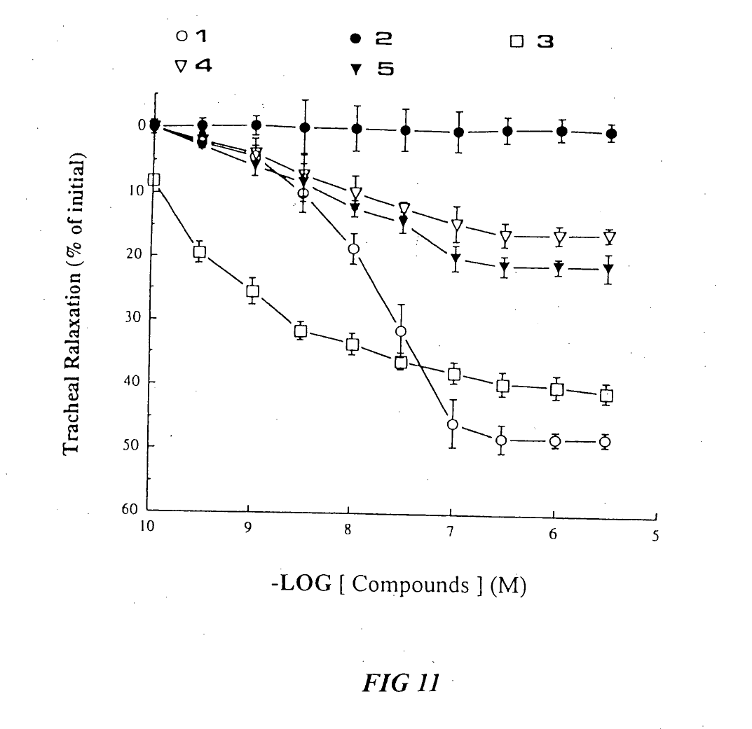

After cumulatively increase the concentrations of compound 1, the changes in the Wistar rat's isolated; reserpine pretreated right atrium beating rate and left atrium isometric contractions were observed. As shown in FIG. 9 and FIG. 10, the cumulative administration of L-isoproterenol would dose-dependently increase the right atrium beating rate and left atrium isometric contractions. Compound 1 could not increase the beating rate and isometric contractions, instead, when the concentration reached 10⁻⁵ M or above, there was blocking effect on beating rate and isometric contractions. Propranolol could retard the beating rate and isometric contractions., and this retardation is proportionally related to the concentration increment. When the dosage concentration is increased to 10⁻⁴→10⁻³ M, it could inactivate or unexcited the tissue. However, at 3×10−4M concentration, atenolol almost does not have atrial blocking effect. Furthermore, when guinea pig's isolated; reserpine pretreated tracheal was cumulatively administered with 10⁻¹⁰˜3×10⁻⁶ M L-isoproterenol or 10⁻¹⁰˜3×10⁻⁶ M Vanidilol; 10⁻¹⁰˜3×10⁻⁶ M nifedipine; and 10⁻¹⁰˜3×10⁻⁶ M compound 1, they could individually caused dose-dependant tracheal relaxation effect.

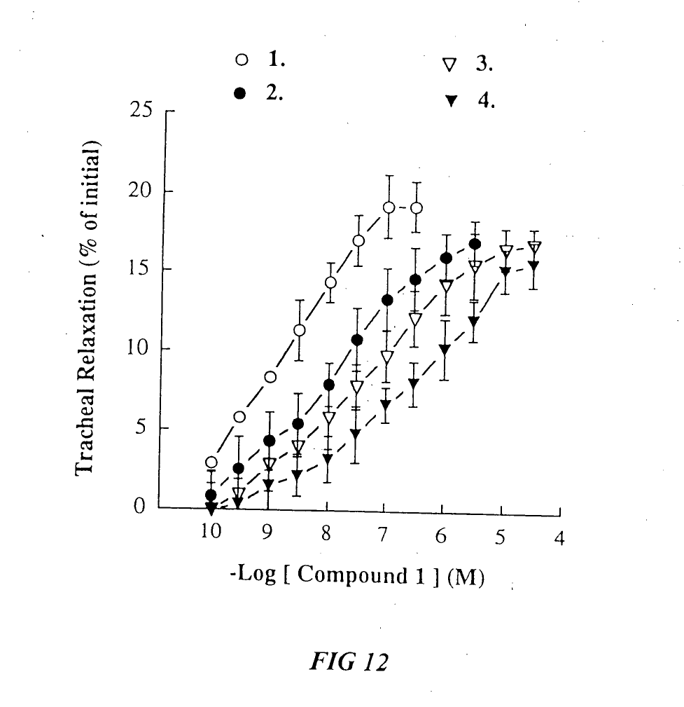

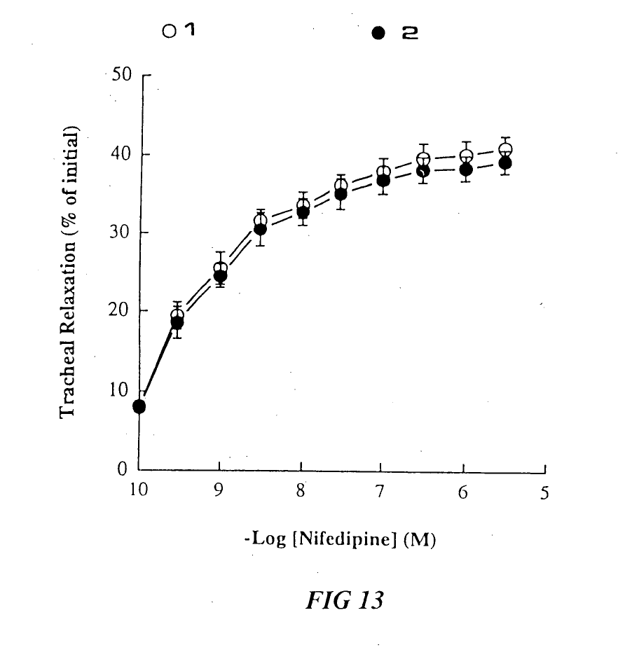

However, as shown in FIG. 10, cumulative administration of propranolol did not produce any relaxation effect. To prove that whether tracheal relaxation effect induced by compound 1 is related to β₂-adrenoceptor, guinea pig's isolated tracheal was pretreated with β₂-adrenoceptor's selective blocking agent, ICI118, 551 at concentrations 10⁻⁸, 10⁻⁹, and 10⁻¹⁰M for 30 minutes. As shown in FIG. 12, the effect of competitive blocking agent, compound 1 had been observed, and the cumulative concentration-dependant curve of compound 1 was concentration-dependently shifting parallel to the right. Furthermore, as shown in FIG. 13, the tracheal relaxation effect of cumulatively administered nifedipine at concentrations 10⁻¹⁰˜3×10⁻⁶ M on guinea pig's isolated and reserpine pretreated tracheal was not blocked by 10⁻⁸ M of ICI 118, 551. This showed that the tracheal relaxation effect produced by nifedipine was not related to β₂-adrenoceptor.

(6) Experiment on the Wistar Rat's Isolated Thoracic Aorta:

After sacrificing Wistar rat of weight between 300-500 gm, the thoracic aorta was immediately removed and placed in cold Kreb's solution. The fatty connecting tissue surrounding the vessel wall was removed and the thoracic aorta was cut into rings of length 5 mm. The two ends of each ring was pieced and fixed with “Z” shaped platinum wires. Then the thoracic aorta was suspended in 10 ml of tissue bath, aerated with air mixture (95% O₂+5% CO₂) and maintained at 37° C., where one end was fixed at the bottom of tissue trough, the other end connected to force transducer to record the long contraction via recorder. The sample was given 1 gm of tension and equilibrated for 60 minutes before the following experiments were carried out:

(a) The effect of compound 1 on the thoracic aorta contraction caused by 75 mM KCl

After thoracic aorta had reached equilibrium in the tissue trough, the normal Kreb's solution in the trough was replaced with 75 mM KCl solution to induce vasoconstriction. When the results had been recorded for at least 35 minutes, the aorta was repeatedly rinsed with normal Kreb's solution. After the aorta had re-equilibrated and at least rested for 60 minutes, different concentrations (10⁻⁸, 10⁻⁷, 10⁻⁶, and 10⁻⁵M) of compound 1 were separately added. 30 minutes later, 75 mM KCl was added again to induce vasoconstriction. This method was used to compare the differences in contraction induced by 75 mM KCl on thoracic aorta pre-treated and non-treated with compound 1.

Results

Effect of Compound 1 on Isolated Guinea-Pig Aorta:

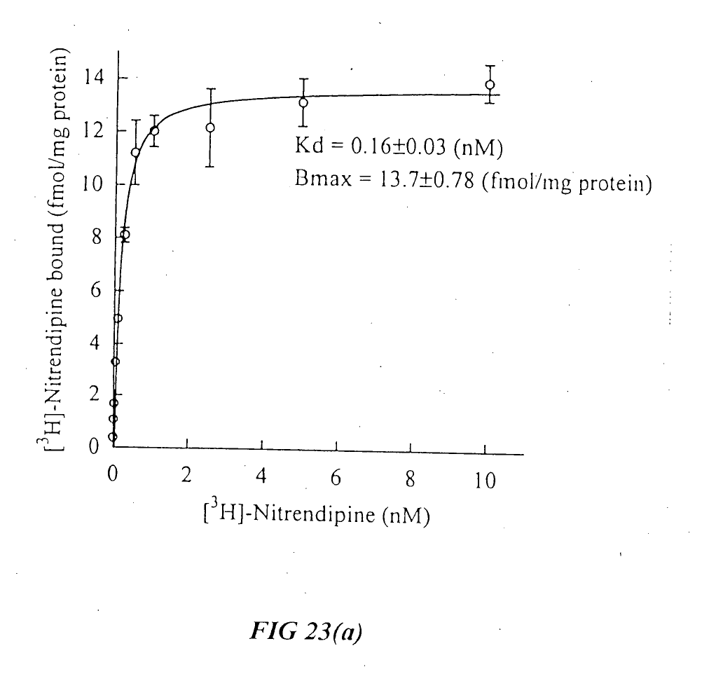

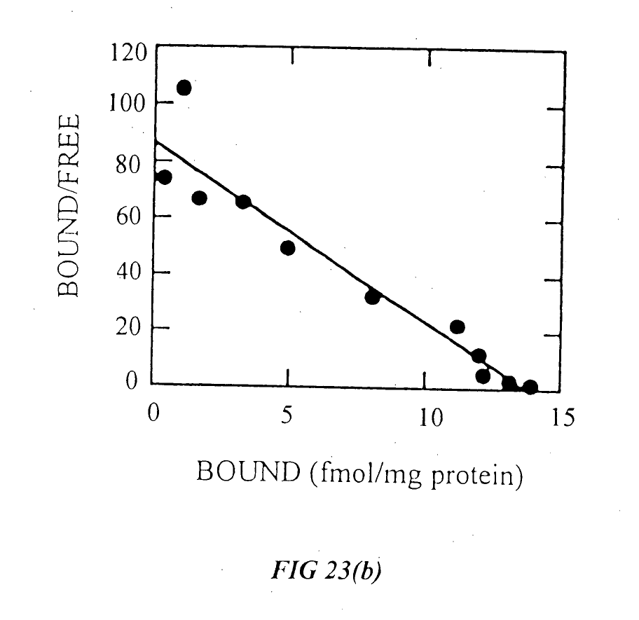

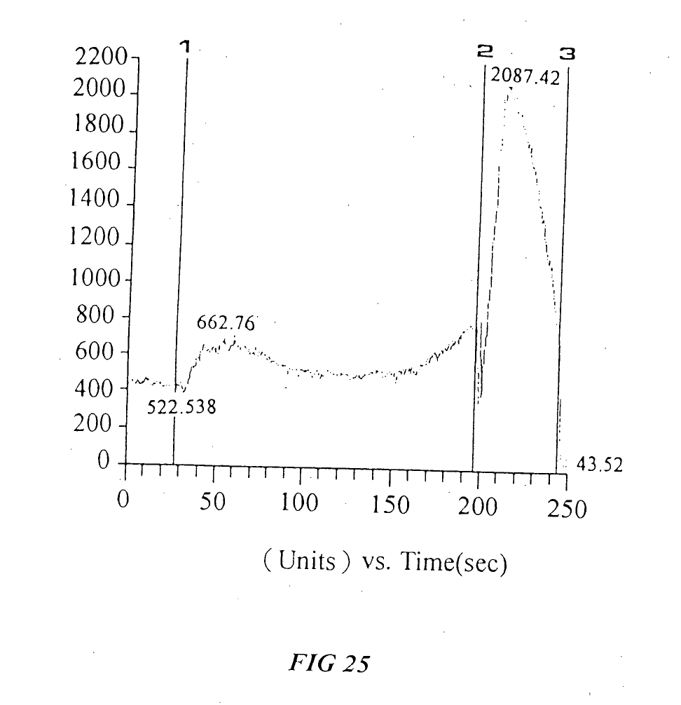

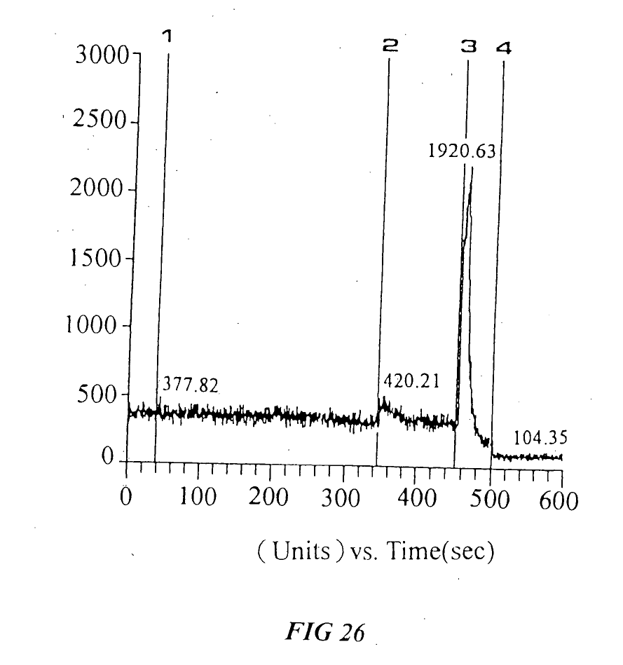

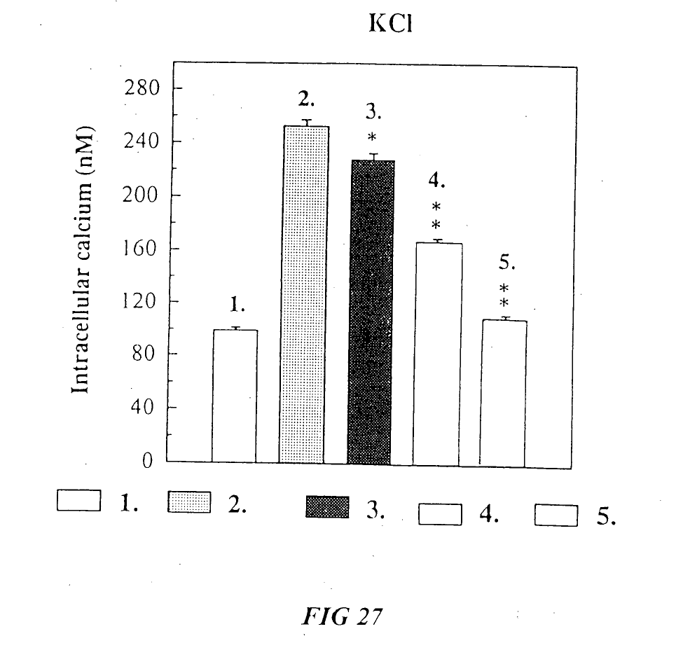

The effect of compound 1 on the thoracic aorta contraction caused by 75 mM KCl. The Wistar rat's isolated thoracic aorta was contraction induced by 75 mM of concentrated Potassium solution. After vasoconstriction had reached stability, different concentrations (10⁻⁸, 10⁻⁷, 10⁻⁶, and 10⁻⁵ M) of compound 1 as stated in FIG. 23 and FIG. 25 were added, which could induce dose-dependant vasoconstriction. Furthermore, the contraction effects of 10−8M of compound 1-9 were indicated on Table 1.

(b) The effect of compound 1 on BayK8644 pretreated thoracic aorta:

0.1 μM of BayK8644 was first added into the tissue trough. 10 minutes later, 75 mM of concentrated Potassium solution was added to induce thoracic aorta contraction. When vasoconstriction had reached stability, different concentrations of (10⁻⁸, 10⁻⁷, 10⁻⁶, and 10⁻5M) compound 1 were separately added. The effects of aorta contraction induced by concentrated Potassium solution were observed.

Results

The Effect of BayK 8644 on Vasorelaxation of Compound 1:

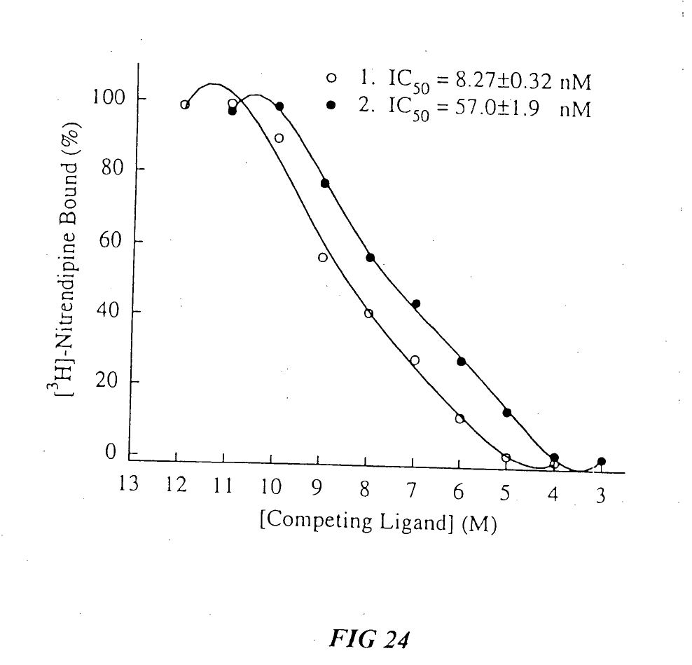

After the thoracic aorta was pre-treated with 0.1 μM of BayK8644 for 10 minutes, concentrated Potassium solution was added to induce vasoconstriction. When the vasoconstriction reached stability, compound 1 of concentrations 10⁻⁸, 10⁻⁷, 10⁻⁶, and 10⁻⁵ M were added. The results was shown in FIGS. 24 and 25 which indicated that pre-treatment with BayK8644 would affect vasoconstriction function of compound 1.

(7) Discussion on Characteristics of Receptor Binding:

(a) Preparation of cell membrane at 4° C.:

The method of Muzzin et. al. (1992) was modified and referenced. The heart and lung of rat were removed and placed in cold Tris buffer. Then the atria and lung were separated and weighed, before placing in cold Tris buffer with volume 20× their weights. Using POLYTRON homogenizer at 15 seconds each time to crushed the tissue for 3˜4 times before homogenization. The homogenized liquid was press filtered through gauze, and the filtered liquid was centrifuged at 700 gm for 12 minutes. The centrifugal fluid was again centrifuged at 10,000 gm for 12 minutes. The second centrifugal fluid was centrifuged for the third time at 29,000 gm for 15 minutes. The pellet finally obtained was re-suspended in Tris buffer as little as possible. Then the method of Brodford (1976) was adopted, where BSA was used as a standard, and protein assay dye was used to determine the protein content in the membrane. Finally, the protein concentration was diluted with Tris buffer to maintain 200˜250, g protein per 100 μl.

| | |
|---|---|
| Tris buffer | pH 7.4 |
| Sucrose | 250 mM |
| Tris buffer | 50 mM |
| MgCl₂ | 1 mM |

(b) Binding assay on receptor:

The methods of Porzig et. al. (1982); Petrus (1988); and Muzzin et. al. (1974) were adopted with modifications. 100 μl of membranes; 50 l of [³H]CGP-12177; 50 μl of test compound in various concentrations; eg. propranolol, compound 1, and Vanidilol were mixed to obtain a final volume of 250 μl. This mixture was placed under 25° C. vibration and reacted for 60 minutes. After reaction, 1 ml of cold Tris buffer was added to terminate the binding reaction. Then Millipore filtration manifold and Whatman GF/C glass fiber were used for rapid press filtration, and 5 ml of cold Tris buffer was used to rinse the filtrate three times. After the filter paper with the filtrate was dried in a 60° C. oven for 3 hours, 5 ml of scintillation fluid was added, and Beckman LS6500 rackbeta liquid scintillation counter was used to determine the strength of radioactivity.

(8) Discussion on the Characteristics of Calcium Ion Receptor Binding:

(a) Preparation of cell membrane at 4° C.:

The method of Tamazawa et. al. (1986) was modified and referenced. The cerebral cortex of rat was removed and placed in cold 0.85% NaCl solution. Then the cortex was weighed, before placing in cold 50 mM Tris-HCl solution and 10 mg EDTA (pH 7.7) with volume 9× its weight. Using POLYTRON homogenizer at 15 seconds each time to crushed the tissue for 3˜4 times before homogenization. The homogenized liquid was press filtered through gauze, and the filtered liquid was centrifuged at 900 gm for 10 minutes. The centrifugal fluid was again centrifuged at 29,000 gm for 15 minutes. The pellet finally obtained was rinsed with 50 mM Tris-HCl solution and 10 mg EDTA (pH 7.7) twice before resuspended in similar Tris-HCl solution and store at −80° C. Then the method of Brodford (1976) was adopted, where BSA was used as a standard, and protein assay dye was used to determine the protein content in the membrane. Finally, the protein concentration was diluted with Tris-HCl solution to maintain at 4 mg/ml.

(b) Binding assay on receptor:

The methods of Gould et. al. (1982) was adopted with modifications. 100 μl of [³H]nitrendipine, 100 μl of Tris-HCl solution, 200 μl of membranes, 100 μl of test compound in various concentrations, propranolol; compound 1, and Vanidilol were mixed to obtain a final volume of 500 μl. This mixture was placed under. 25° C. vibration and reacted in the dark for 60 minutes. Then Millipore filtration manifold and Whatman GF/C glass fiber were used for rapid press filtration, and 4 ml of cold Tris-HCl solution and 0.1 mM EDTA (pH 7.7) were used to rinse the filtrate four times. After the filter paper with the filtrate was dried in a 70° C. oven for 1 hours, 4 ml of scintillation fluid was added, and Beckman LS6500 rackbeta liquid scintillation counter was used to determine the strength of radioactivity.

(9) Fluorescence Determination of Intracellular Calcium Ion:

(a) Cell Culture

Originated from American type culture collection, CRL 1446, clonal cell line A7r5 from Rockville, Md. was cultured in Dulbecco's modified Eagle's medium (DMEM) supplemented with 10% fetal bovine serum; 10000 U/ml penicillin G and 10 mg/ml streptomycin, and placed in 37° C. incubation oven with 95% O2+5% CO₂ aeration. Cells were subcultured weekly after detachment by using culture medium containing 1% trypsin. This stage of experiment was completed after cells reached confluence.

(b) Measurement of [Ca²⁺ ]ᵢ concentration of A7r5

The cells were prepared as mentioned above. The concentration of [Ca²⁺ ]i was measured using a fluorescent indicator, fluo 3-AM, where the cells were scanned by image and line scan. Firstly, in A7r5 cell culture medium, 5 μm of fluo 3-AM, Calbiochem USA was added to stain the cells. After incubating in 37° C. of CO2 for an hour, Tyrode's solution containing calcium ions were used to rinse the cells for three times to remove excess extra-cellular Fluo 3-AM. A laser cytometer from Meridian Instruments Inc., USA) was used to measure the changes in the concentration of [Ca²⁺ ]ᵢ. Firstly, phase optics microscope was used to locate individual cells, then the fluorescence intensity of intracellular fluo 3-AM was determined by laser fluorescence scanning. The intensity f fluo 3-AM was measured by line scanning at 30 second interval with an argon laser at 488 mm. A computer was used to convert the fluorescent line into a pseudogrey level line, coded according to fluorescence intensity. After addition of 50 mM KCl and scanned for 125 seconds to establish a baseline, the cells were rinsed with Tyrode's solution, 10⁻⁸, 10⁻7, and 10⁻⁶ M of compound 1 were separately added and each scanned for 300 seconds. Finally, 50 mM KCl was added to record the changes in the concentration of [Ca²⁺]ᵢ.

The fluorescence ratio and concentration of [Ca²⁺]ᵢ are proportionately related. The relationship between the above mentioned two items could be represented by a equation
[Ca2+]=Kd(F−Fmix) Fmax−F
Kd is the dissociation constant 320 nM for fluo 3-AM. Adding 10⁻⁴M of nonfluoroscent Ca²⁺ ionophore 4-bromo-A-23187 to the cells could obtain the Fmax value, while further addition of 5 mM EDTA to remove intra and extra-cellular calcium ions, the Fmin could be obtained. Furthermore, using 10 μM of BayK8644, a Ca2+ channel activator to replace the previous KCl, and with similar procedure for different concentrations (10⁻⁸, 10⁻⁷, and 10⁻⁶ M) to observe the blocking effect of compound 1 on intracellular Ca²⁺ concentration increment by BayK8644 (10 μM).

Results

Compound 1 on β-Receptor Binding

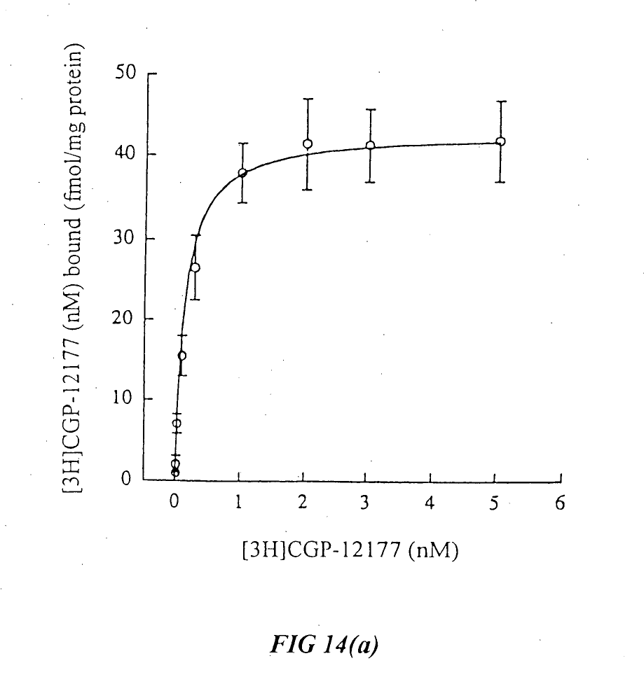

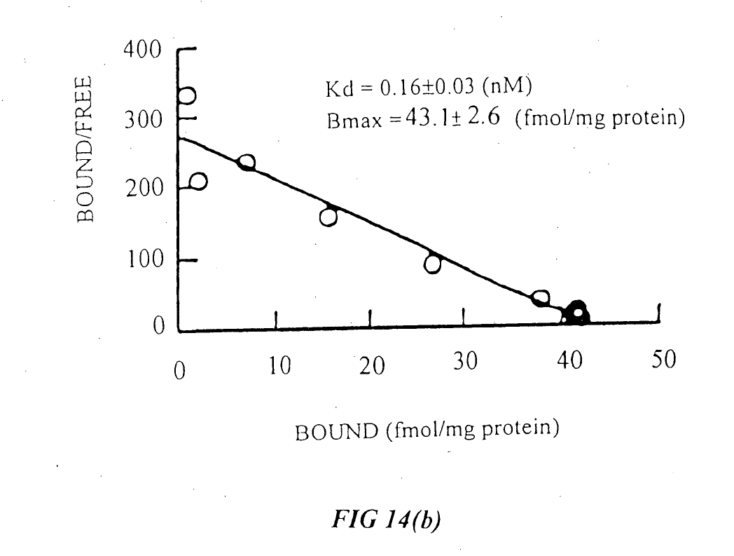

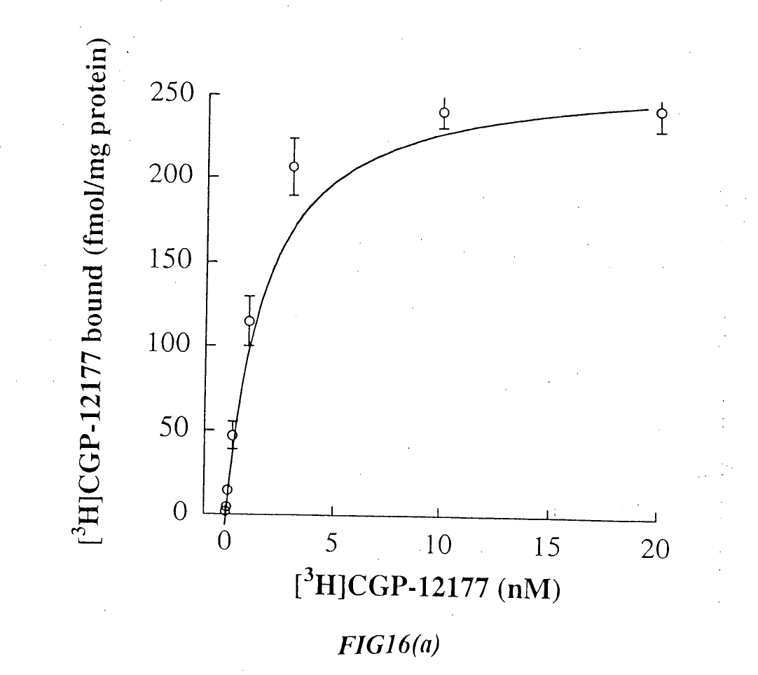

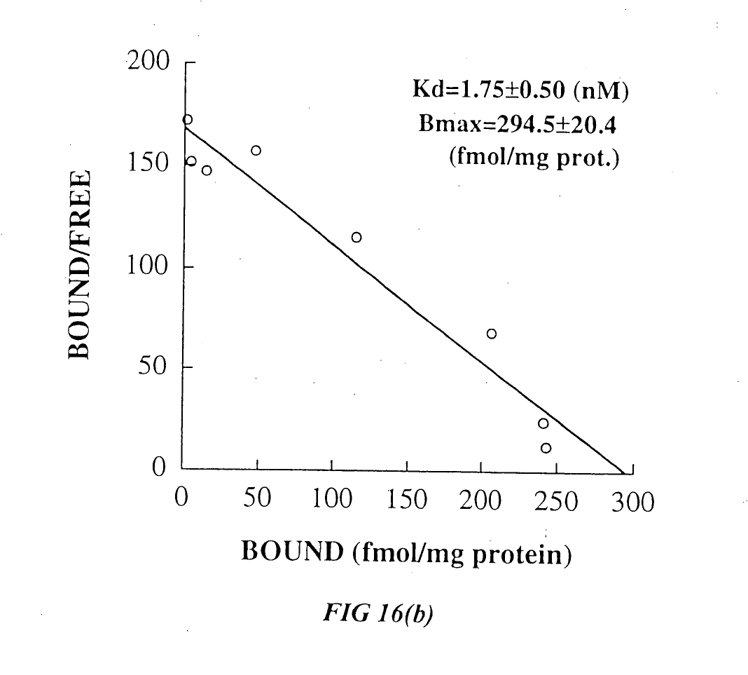

As shown in FIGS. 14 and 16, using different concentrations from 0.003˜80 nM of [3H]CGP-12177 on dose-dependant binding research indicated that [3H]CGP-12177 binding to Wistar rat's atria and lung membrane could reach saturation. Furthermore, as shown in FIGS. 14 and 16, analysis method by Scatchard (1949) was used to determine receptor affinity and number of binding position. At 25° C., the equilibrated Kd of atria and lung membranes were 0.16±0.03 and 1.75±0.50 (nM) respectively. The maximum binding density (Bmax) of receptor were 43.1±2.6, 294.5±20.4 (fmol/mg protein). These data indicated that the equation is average±standard error. The receptor binding of [3H]CGP-12177 could reach stability in approximately 20 minutes and could last up to 90 minutes.

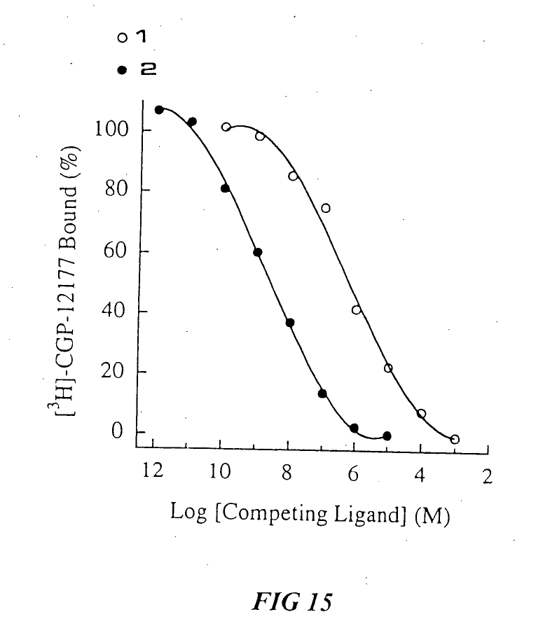

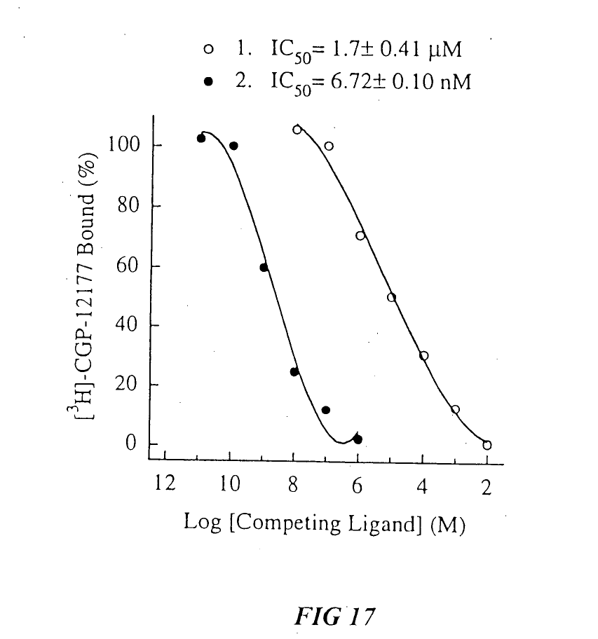

As shown in FIGS. 15 and 17, the pKi values of competitive curve of β-adrenoceptor blocking agent on Wistar rat's atrium; lung membrane receptors were listed on Table 2. For β-adrenoceptor blocking agent in blocking the binding of [3H]CGP-12177 to atrial β 1-receptor; lung β 2-receptor, its effectiveness in sequence is propranolol>>compound 1.

Compound 1 on Calcium Ion Blocking:

(a) The effect of compound 1 on atrial beating rate changes induced by CaCl₂:

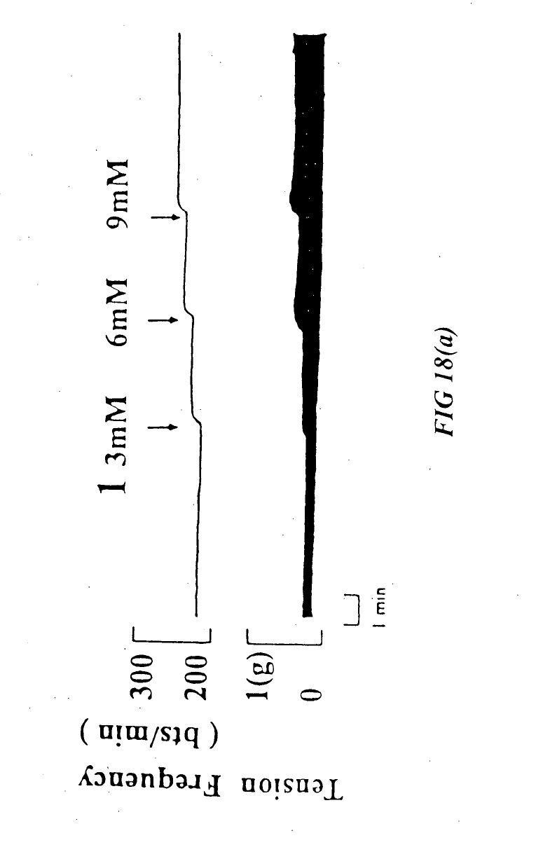

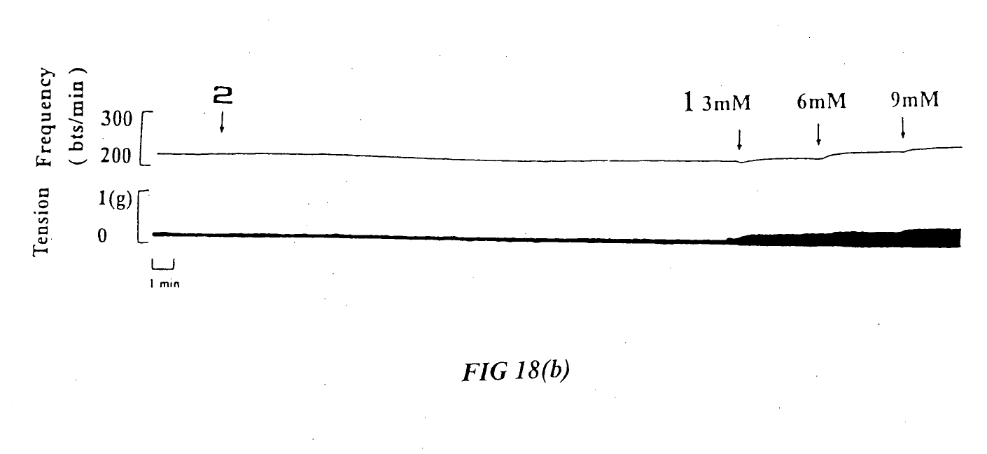

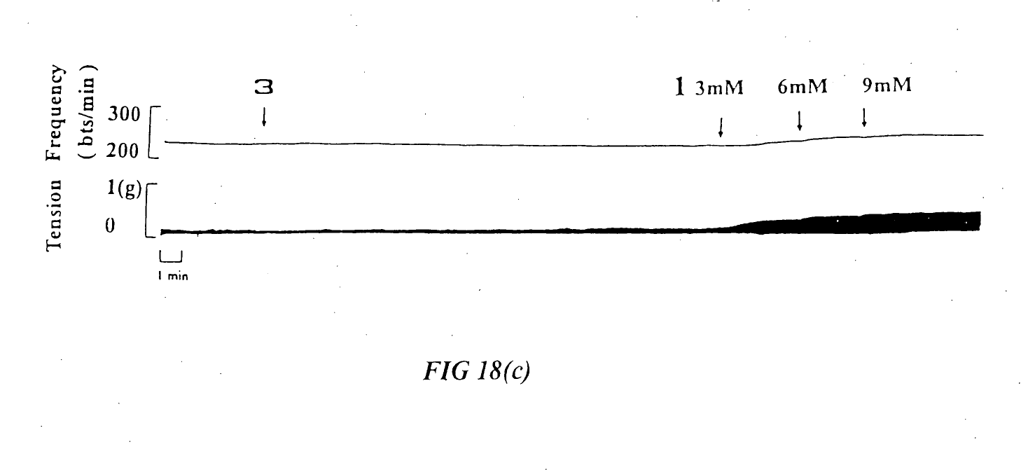

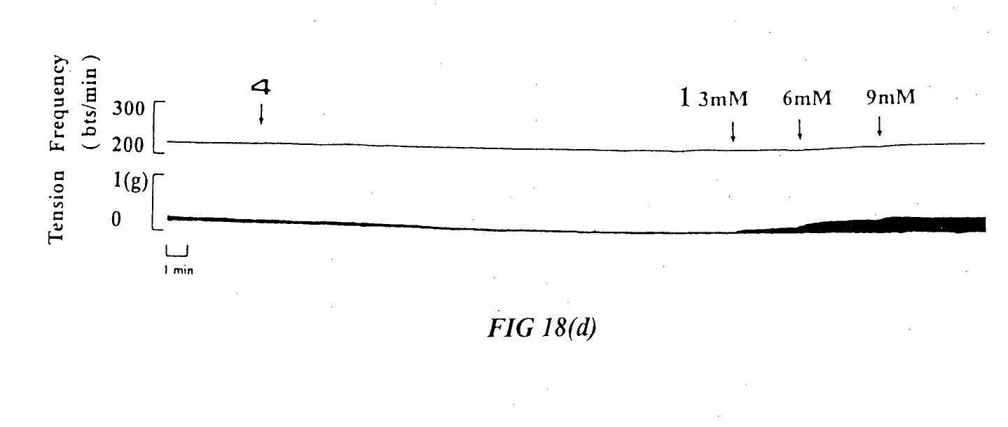

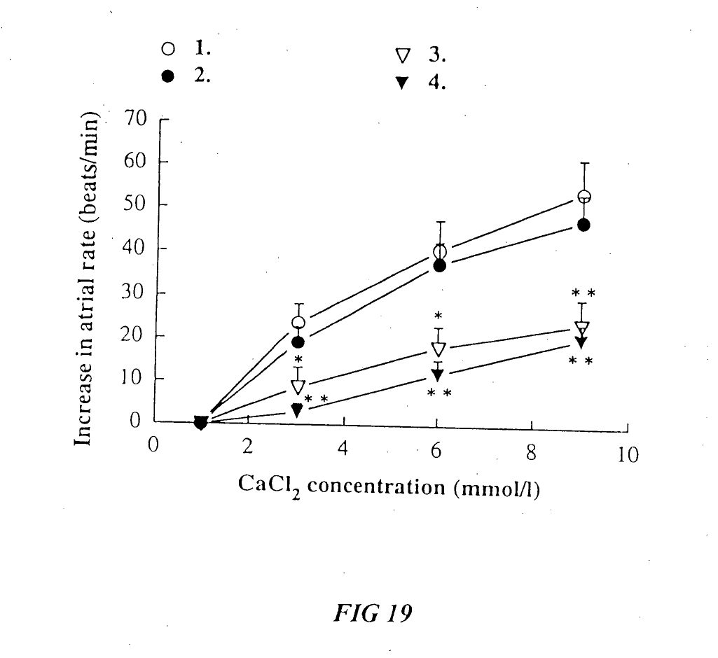

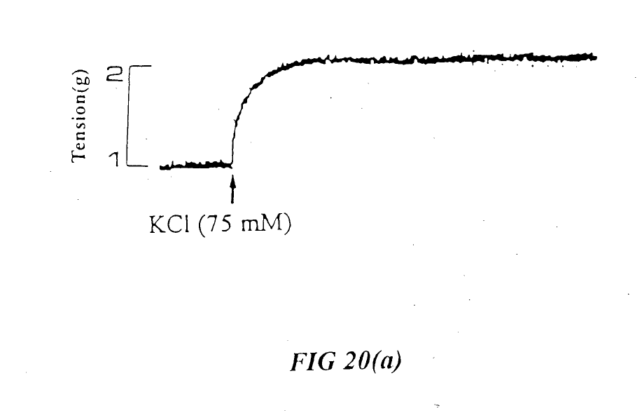

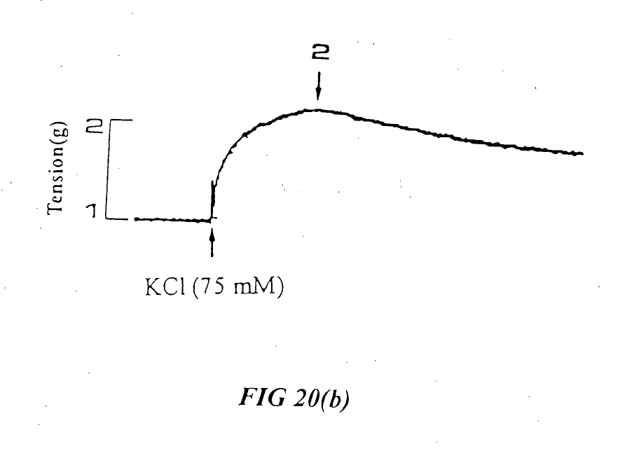

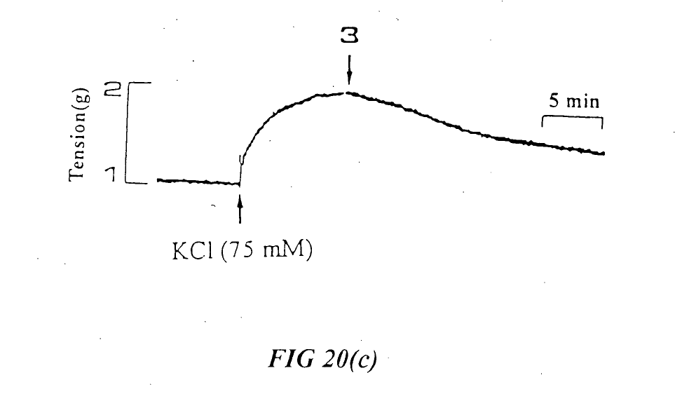

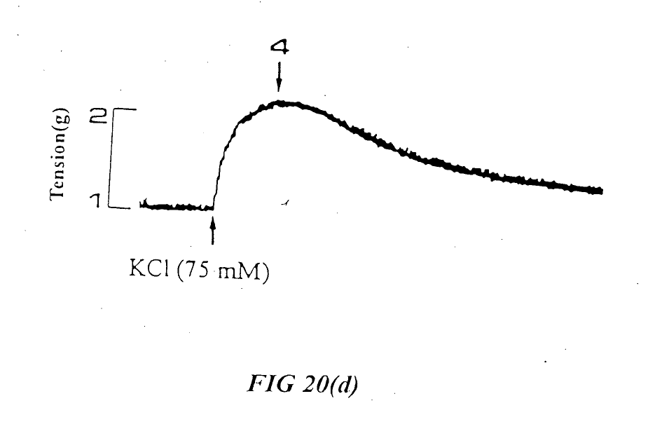

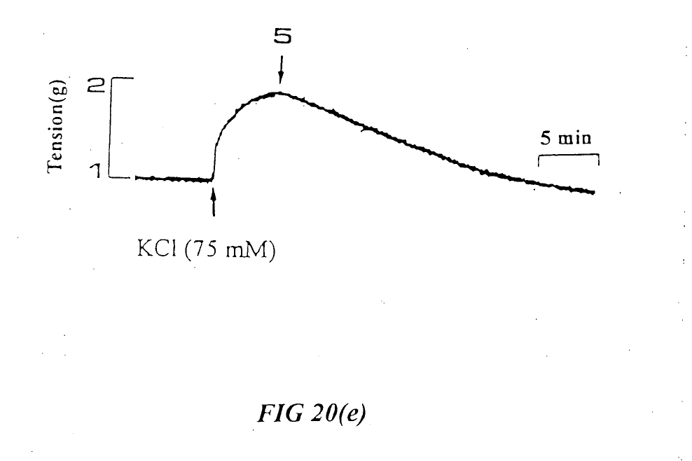

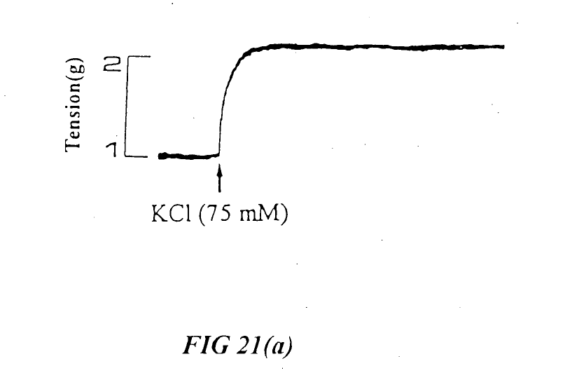

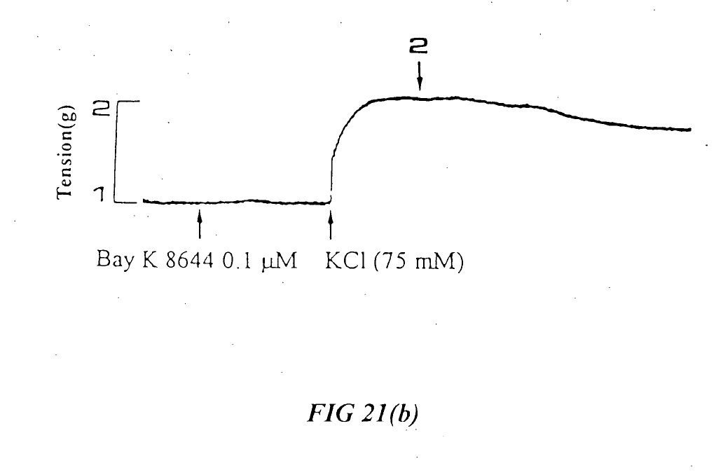

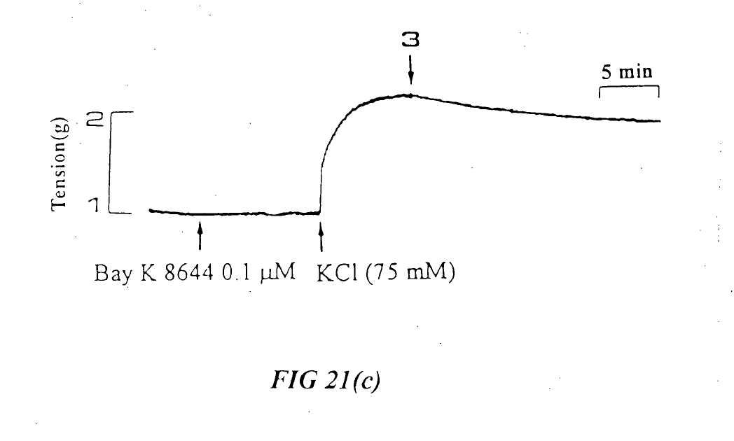

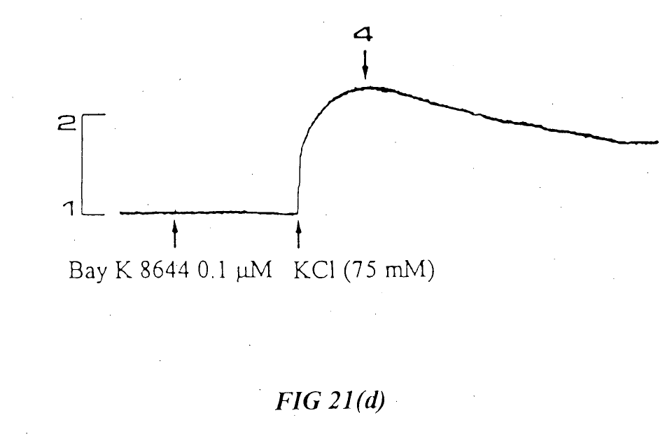

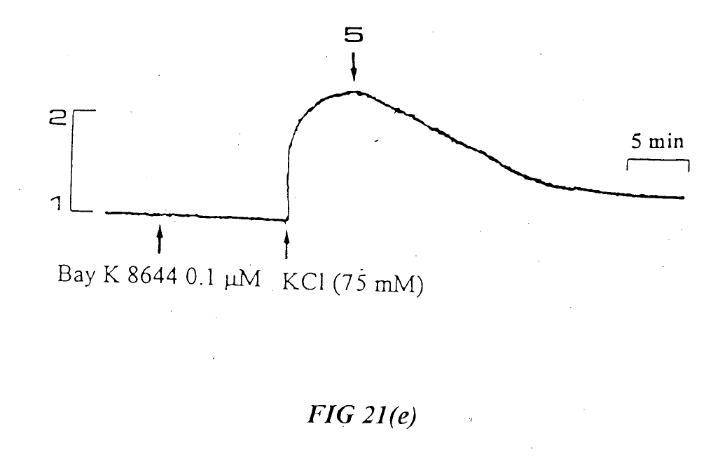

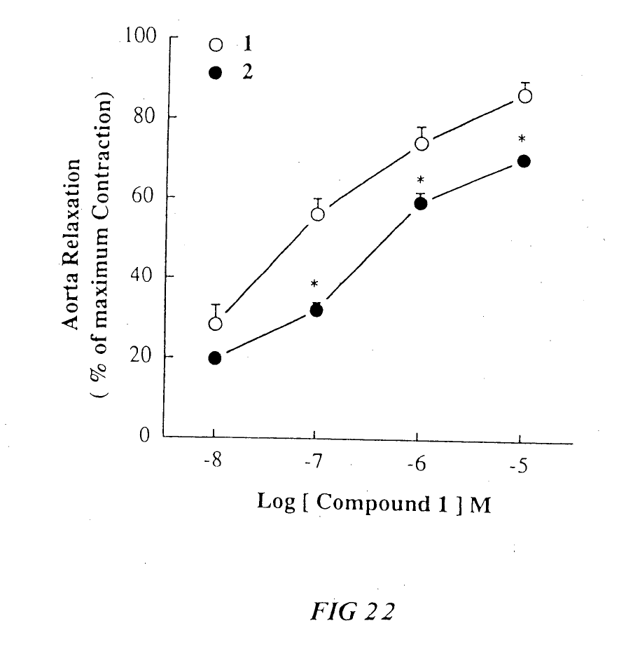

Cumulatively administered 3.0; 6.0; and 9.0 mM of CaCl₂ into Kreb's solution showed that atrial-beating rate had a concentration dependant increment when 3.0; 6.0; and 9.0 mM of CaCl₂ were separately added. As shown in FIGS. 18, 22, under the presence of different concentrations (10⁻⁷, 10⁻⁶, and 10⁻5M) of compound 1, increased atrial beating rate effect induced by cumulatively administered 3.0, 6.0, and 9.0 mM of CaCl₂ was found to be blocked, that is, under similar concentrations of CaCl₂, the atrial beating rate could not reach similar strength of increment.

(b) Compound 1 on binding research of calcium ion receptor

As shown in FIG. 23, using different concentrations (0.001˜10 nM) of [³H]nitrendipine in dose dependant receptor binding research indicated that [³H]nitrendipine binding to rat's cerebral cortex could reach saturation. Furthermore, as shown in FIGS. 14 and 16, analysis method by Scatchard (1949) was used to determine receptor affinity and number of binding position. At 25° C., the equilibrated Kd of atria and lung membranes were 0.16±0.03 and 1.75±0.50 (nM) respectively. The maximum binding density (Bmax) of receptor were 43.1±2.6, 294.5±20.4 (fmol/mg protein). These data indicated that the equation is average±standard error. The receptor binding of [³H]CGP-12177 could reach stability in approximately 20 minutes and could last up to 90 minutes.

As shown in FIG. 24, the pKi values of competitive curve of calcium ion entry blocking agent on Wistar rat's cerebral cortex calcium ion were listed on Table 3. For calcium ion entry blocking agent in blocking the binding of [³H]nitrendipine to cerebral cortex calcium ion receptors, its effectiveness in sequence is nifedipine>compound 1.

(c) Fluoro-measurement of intracelluar calcium ion:

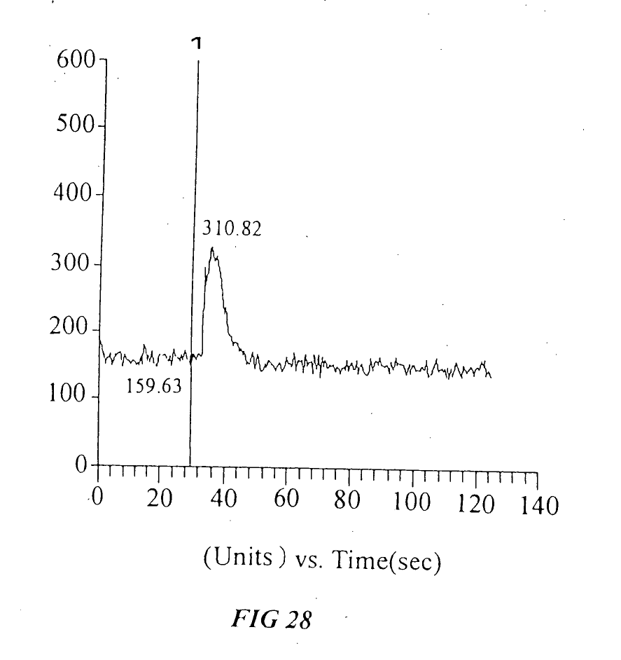

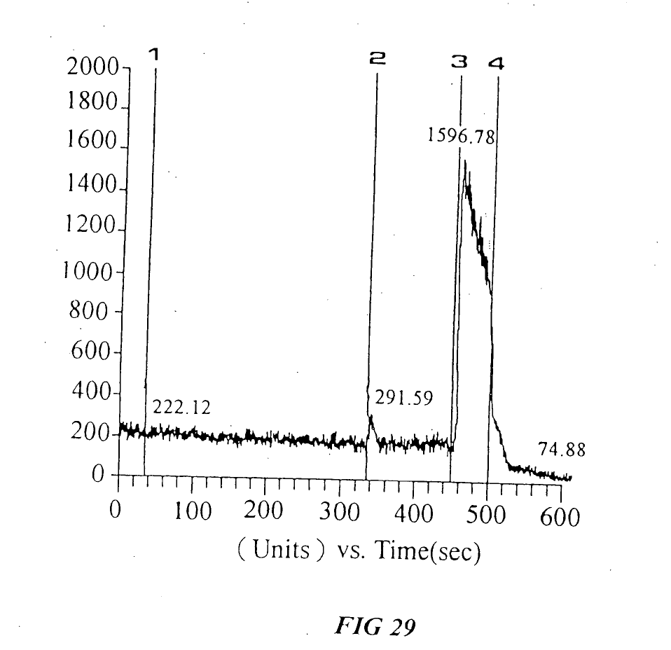

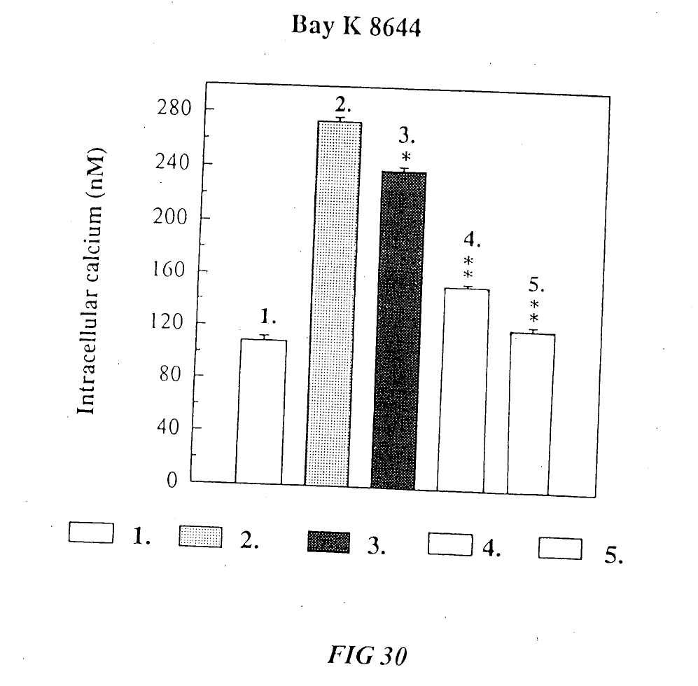

Separately adding 50 mM KCl and 10 μM BayK8644 to A7r5 cells stained with fluo 3-AM, then the changes in the concentration of intracellular calcium ion were measured by fluoro-measurement method. As shown in FIGS. 25; 28, the results indicated that both KCl and BayK8644 could induce influx of calcium ions into A7r5 cells, thus increase the concentration of intracellular calcium ions, producing a high peak for [Ca²⁺ ]₁. Next, the cells were separately treated with 10⁻⁸, 10⁻⁷, and 10⁻⁶M of compound 1 for 5 minutes, before addition of KCl or Bay8644. As shown in FIGS. 29; 30, the results indicated that the calcium ion influx effect of both KCl and BayK8644 had been significantly blocked, such that at this period of time there was no significant peak for [Ca²⁺ ]ᵢ. Among which, the blocking effect of compound 1 in preventing increment of calcium ion concentration is greater for BayK8644 than for KCl, also this blocking effect of compound 1 showed a concentration dependant curve.

### EXAMPLE 1

### Synthesis of Compound 1

8 gm of sodium hydroxide was dissolved in 100 ml of absolute alcohol. 1 molar of 4-hydroxy-3-methoxy-1-benzaldehyde was dissolved in the above prepared solution and mixed under room temperature. Then 5 molar of Epichlorohydrin was added and reacted under room temperature. TLC was used to ensure complete reaction. After decompression to concentrate, the concentrated liquid was separated by silica gel filled column. Hexane:Ethylacetate=1:9 was used as the eluent solution to obtain white coarse crystals. Then hexane was used to re-crystallized repeatedly to obtain purified N-[4-(2,3-epoxypropoxy)-3-methoxy]-1-benzaldehyde.

Similar molar of N-[4-(2,3-epoxypropoxy)-3-methoxy]-1-benzaldehyde and tert-butylamine were dissolved in 100 ml of absolute alcohol, and undergone amination in slight warmth. After mixing and left overnight, a white solid crystal could be obtained. Using methanol to re-crystallized, purified compound N-{4-[2-hydroxy-3-(tert-butylamino)propoxy]-3-methoxy}-1-benzaldehyde was obtained.

0.01M of N-{4-[2-hydroxy-3-(tert-butylamino)propoxy]-3-methoxy}-1-benzaldehyde was heated with 2.4 ml (0.02M) of methylacetoacetate; 15 ml ethanol; and 10 ml of concentrated amine solution, and reacted in 55° C. water bath for 15 hours. The solution obtained from the reaction was directly decompressed to concentrate and dehydrated to remove ethanol. The remaining solution was added with 50 ml of saturated NaCO₃ solution, and extracted repeatedly with CHCl₃ and water. All the organic layers obtained were dried; filtered and concentrated. The oil layer obtained was combined with Ethanol-HCl mixed liquid and purified by column chromatography (methanol:Ethylacetate=3:7). Slightly yellowish white crystals were extracted by ethylacetate, then methanol: Ethylacetate=1:9 was used for repeated re-crystallized to obtain purified compound 1.

The structure of compound obtained is C₂₅H₃₆O₇N₂.HCl, and the molecular weight through mass spectrometer is 513. ¹H-NMR (CDCl₃) δ:1.46 (s, 9H, 3XCH₃), 2.33 (s, 6H, 2XCH₃), 3.00-3.45 (m, 2H, CH₂—NH), 3.65(s, 6H,CO—OCH3×2), 3.77 (s, 3H, OCH₃), 3.93-4.14 (m, 2H, Ar—OCH₂), 4.53 (m, 1H, CH—OH), 4.94 (s, 1H, Ar—CH<), 5.45 (brs, 1H, replaceable, —NH—), 6.33-6.87 (m, 3H,Ar), 8.33 (brs, 1H, replaceable, CH₂—NH—C), 9.28 (brs, 1H, replaceable, OH); IR (KBr):3360, 2950, 1710, 1655, 1440, 1380 cm⁻1. MS m/s: 513 (Scan FAB+) Anal. (C₂₅H₃₆O₇N₂.HCl)C,H,N. In accordance to the analytical data, compound 1 was found to be 4-{4-[2-hydroxy-3-(tert-butylamino)propoxy]-3-methoxy} phenyl}}-2,6-dimethyl-3,5-dicarbo methoxy-1,4-dihydro-pyridine.

### EXAMPLE 2

### Synthesis of Compound 2

0.2M of 2-methoxyphenol and 0.4M of Ethylene dibromide were heated till boil in a triple-necked flask, and mixed with a rod for 30 minutes. 125 ml of 1.6N sodium hydroxide was added, and heated with mixing until layers were divided. After heating overnight, TLC was used to ensure complete reaction, and CHCl₃ was repeatedly used to extract the organic layer.

300 ml of 2N sodium hydroxide was used to rinsed the organic layer, before anhydrous magnesium sulfate was added and left overnight. Then the compound was filtered, and decompressed to concentrate. Silica gel filled column and hexane:ethylacetate=9:1 as the eluent solution, and the first intermediate product 2-methoxy-1-oxyethylbromide benzene was obtained.

Using similar molar of potassium phthalimide to perform the above-mentioned procedure, 2-methoxy-1-oxyethyl bromide benzene was obtained. This was dissolved in dimethylformamide, and its temperature raised to 55° C. within 5 minutes. After this temperature was maintained for 30 minutes, it was lowered to room temperature, and CHCl₃ was used to repeatedly extract the organic layer of the former. The layer was rinsed with 0.2N sodium hydroxide, then added with anhydrous magnesium sulfate and left to rest overnight to remove residual liquid, before filtration and decompression to concentrate. Finally via crystallization, the second intermediate product, 2-methoxy-1-oxyethylphthalimide benzene was obtained.

Similar molar of 2-methoxy-1-oxyethylphthalimide benzene and hydrazine hydrate were dissolved in absolute alcohol, then brought to boil for 45 minutes. After addition of suitable amount of 18% HCl salt to produce white sediment, the solution was boiled for another hour. The compound was then filtered and rinsed with absolute alcohol. The filtrate was decompressed to concentrate and rinsed in 20% sodium hydroxide. After the organic layer was extracted by CHCl₃, anhydrous magnesium sulfate was added, and the layer was left to rest overnight before second filtration. With decompression to concentrate, 2-methoxy-1-oxyethylamino benzene was obtained.

8 gm of sodium hydroxide was dissolved in 100 ml absolute alcohol 1 molar of 4-hydroxy-3-methoxy-1-benzaldehyde was dissolved in the above mentioned solution. This solution was stirred under room temperature, and 5 molar of epichlorohydrin added to react. TLC was used to determine that the reaction had been completed. Using hexane: ethylacetate=9:1 as the silica column eluent solution, and repeatedly re-crystallized by hexane to obtain purified N-[4-(2,3-epoxypropoxy)-3-methoxy]-1-benzaldehyde.

Similar molar of 2-methoxy-1-oxyethylamino benzene and compound prepared from the above mentioned procedure were dissolved in 100 ml of absolute alcohol, and undergone amination in slight warmth. After stirring and left to rest overnight, a yellowish white solid crystal was obtained. Using methanol to re-crystallized, a purified compound N-{4-[2-hydroxy-3-(2-methoxy-1-oxyethylaminobenzene) propoxy]-3-methoxy)-1-benzaldehyde was obtained.

0.01M of N-{4-[2-hydroxy-3-(2-methoxy-1-oxyethylaminobenzene) propoxy]-3-methoxy}-1-benzaldehyde was dissolved in a solution containing 2.4 ml (0.02M) methylacetoacetate, 15 ml ethanol and 10 ml concentrated ammonia water; heated and placed in a water bath of 55° C. for 15 hours. After the reaction, the solution obtained was directly decompressed to concentrate and ethanol removed. 50 ml of saturated NaCO₃ solution was added to the remaining solution and the organic layers were extracted by CHCl₃ and water for several times. All the organic layers obtained were dehydrated; filtered and concentrated, then purified by column chromatography (methanol:ethylacetate=1:9) to obtain purified compound 2.

The structure of compound obtained, compound 2 is C₃₀H₃₈O₉N₂, and the molecular weight through mass spectrometer is 570. ¹H-NMR(CDCl₃) δ:2.26(s, 6H, 2XCH₃), 2.50-2.89 (m, 4H, CH₂—NH—CH₂), 3.56 (s, 6H, 2XCO—OCH₃), 3.68-3.74 (d, 6H, 2xOCH₃), 3.86-4.05 (m, 4H,2×Ar—OCH₂), 4.4 (m, 1H, CH—OH), 4.83(s, 1H, Ar—CH<), 6.84-7.37(m, 7H, Ar—H), 8.33 (s, 1H, replaceable, NH—). MS m/s: 570 (Scan FAB⁺) Anal. (C₃₀H₃₈O₉N₂)C,H,N. In accordance to the analytical data, compound 2 was found to be 4-{{N-{4-[2-hydroxy-3-(2-methoxy-1-oxyethyl-aminobenzene)propoxy]-3-methoxy}phenyl}}-2,6-dimethyl-3,5-dicarbo-methoxy-1,4-dihydropyridine.

### EXAMPLE 3

### Synthesis of Compound 3

8 gm of sodium hydroxide was dissolved in 100 ml absolute alcohol. 1 molar of 4-hydroxy-3-methoxy-1-benzaldehyde was dissolved in the above mentioned solution. This solution was stirred under room temperature, and 5 molar of epichlorohydrin added to react. TLC was used to determine that the reaction had been completed. Using hexane:ethylacetate=1:9 as the eluent solution of silica gel column, and repeatedly re-crystallized by hexane to obtain purified N-[4-(2,3-epoxypropoxy)-3-methoxy]-1-benzaldehyde.

Similar molar of N-[4-(2,3-epoxypropoxy)-3-methoxy]-1-benzaldehyde and 2-methoxy-1-oxyethylaminobenzene were dissolved in 100 ml of absolute alcohol, and undergone amination in slight warmth. After mixing and left overnight, a white solid crystal could be obtained. Using methanol to re-crystallized, purified compound N-{4-[2-hydroxy-3-(2-methoxy-1-oxyethylamino-benzene) propoxy]-3-methoxy}-1-benzaldehyde was obtained.

0.01M of N-{4-[2-hydroxy-3-(2-methoxy-1-oxyethylaminobenzene)propoxy]-3-methoxy}-1-benzaldeh yde was dissolved in a solution containing 2.4 ml (0.02M) ethylacetate, 15 ml ethanol and 10 ml concentrated ammonia water; heated and placed in a water bath of 55° C. for 15 hours. After the reaction, the solution obtained was directly decompressed to concentrate and ethanol removed. 50 ml of saturated NaCO₃ solution was added to the remaining solution and the organic layers were extracted by CHCl₃ and water for several times. All the organic layers obtained were dehydrated; filtered and concentrated, then the oil layer obtained was added to a Ethanol-HCl mixed solution, and purified by column chromatography (methanol:ethylacetate=3:7). Ethylacetate was first used to precipitate out pale yellowish white crystals, then ethanol:ethylacetate=1:9 was used to repeatedly re-crystallized to obtain purified compound 3.

The structure of compound obtained, compound 3 is C₃₂H₄₂O₉N₂, and the molecular weight through mass spectrometer is 598. ¹H-NMR(CDCl₃) δ:1.20-1.27(t, 6H,2XOCH₂CH₃), 2.33(s, 6H, 2×CH₃), 2.50-2.89(m, 4H, CH₂—NH—CH₂), 3.45(m, 4H, 2xCO—OCH₂CH₃), 3.78-3.84(d, 6H,2×OCH₃), 4.95(s, 1H, Ar—CH<), 5.73 (brs, 1H, replaceable, —NH—), 6.76-6.91(m, 7H, Ar). MS m/s: 598(Scan FAB+) Anal. (C₃₂H₄₂O₉N₂)C,H,N. In accordance to the analytical data, compound 1 was found to be 4-{{N-{4-[2-hydroxy-3-(2-methoxy-1-oxyethyl-aminobenzene)propoxy]-3-methoxy) phenyl}}-2,6-dimethyl-3,5-dicarboethoxy-1,4-dihydropyridine.

### EXAMPLE 4

### Synthesis of Compound 4

8 gm of sodium hydroxide was dissolved in 100 ml absolute alcohol. 1 molar of 5-chlorosalicylaldehyde was dissolved in the above mentioned solution. This solution was stirred under room temperature, and 5 molar of epichlorohydrin added to react. TLC was used to determine that the reaction had been completed. Using hexane: ethylacetate=1:9 as the eluent solution of silica gel column, and repeatedly re-crystallized by hexane to obtain purified N-[2-(2,3-epoxy-propoxy)-5-chloro]-1-benzaldehyde.

Similar molar of N-[2-(2,3-epoxypropoxy)-5-chloro]-1-benzaldehyde and tert-butylamine were dissolved in 100 ml of absolute alcohol, and undergone amination in slight warmth. Using column chromatography (methanol: ethylacetate=1:1) to purify, a white solid crystal could be obtained. Using methanol to re-crystallized, purified compound N-{2-[2-hydroxy-3-(tert-butylamino)propoxy]-5-chloro}-1-benzaldehyde was obtained.

0.01M of N-{2-[2-hydroxy-3-(tert-butylamino)propoxy]-5-chloro}-1-benzaldehyde was dissolved in a solution containing 2.4 ml (0.02M) ethylacetoacetate, 15 ml ethanol and 10 ml concentrated ammonia water; heated and placed in a water bath of 55° C. for 15 hours. After the reaction, the solution obtained was directly decompressed to concentrate and ethanol removed. 50 ml of saturated NaCO₃ solution was added to the remaining solution and the organic layers were extracted by CHCl₃ and eater for several times. All the organic layers obtained were dehydrated; filtered and concentrated, then the oil layer obtained was added to a Ethanol-HCl mixed solution, and purified by column chromatography (methanol:ethylacetate=3:7). Ethylacetate was first used to precipitate out pale yellowish white crystals, then ethanol ethylacetate=1:9 was used to repeatedly re-crystallized to obtain purified compound 4.

The structure of compound obtained, compound 4 is C₂₆H₃₇O₆N₂Cl, and the molecular weight through mass spectrometer is 508.5. ¹H-NMR(CDCl₃) δ:1.15-1.27 (s, 9H, 3XCH₃), 1.36(m, 6H, 2×CO—OCH₂CH₃), 2.29-2.30(d, 6H,2×CH₃), 2.69-2.98 (m, 2H, CH2—NH), 3.46-3.76 (s, 4H,2×CO—OCH₂CH₃),3.88-4.23 (m,2H,Ar—OCH₂), 4.27 (s, 1H,CH—OH), 5.21(s, 1H, Ar—CH<), 5.74 (brs, 1H, replaceable, —NH—), 6.67-7.54(m, 3H, Ar). MS m/s: 508.5 (Scan FAB+) Anal. (C₂₆H₃₇O₆N₂Cl)C,H,N. In accordance to the analytical data obtained from chemical experiments, compound 4 was found to be 4-{{2-[2-hydroxy-3-(tert-butylamino)propoxy]-5-choloro} phenyl}}-2,6-dimethyl-3,5-dicarbo-ethoxy-1,4-dihydropyridine.

### EXAMPLE 5

### Synthesis of Compound 5

0.8 gm of sodium hydroxide was dissolved in 100 ml absolute alcohol. 1 molar of 5-chlorosalicylaldehyde was dissolved in the above mentioned solution. This solution was stirred under room temperature, and 5 molar of epichlorohydrin added to react. TLC was used to determine that the reaction had been completed. After decompressed to concentrate, the solution was separated by silica gel column, with hexane: ethylacetate=1:9 as the eluent solution. A white coarse crystal was obtained. With repeated re-crystallization by hexane to obtain purified N-[2-(2,3-epoxypropoxy)-5-chloro]-1-benzaldehyde.

Similar molar of N-[2-(2,3-epoxypropoxy)-5-chloro]-1-benzaldehyde and 2-methyl-1-oxyethylaminobenzene were dissolved in 100 ml of absolute alcohol, and undergone amination in slight warmth. Using column chromatography (methanol ethylacetate=1:1) to purify, a white solid crystal could be obtained. Using methanol to re-crystallized, purified compound N-{2-[2-hydroxy-3-(n-butylamine)propoxy]-5-chloro}-1-benzaldehyde was obtained.

0.01M of N-{2-[2-hydroxy-3-(n-butylamine)propoxy]-5-chloro}-1-benzaldehyde was dissolved in a solution containing 2.4 ml (0.02M) ethylacetoacetate, 15 ml ethanol and 10 ml concentrated ammonia water; heated and placed in a water bath of 55° C. for 15 hours. After the reaction, the solution obtained was directly decompressed to concentrate and ethanol removed. 50 ml of saturated NaCO₃ solution was added to the remaining solution and the organic layers were extracted by CHCl₃ and water for several times. All the organic layers obtained were dehydrated; filtered and concentrated, then the oil layer obtained was added to a Ethanol-HCl mixed solution, and purified by column chromatography (methanol ethylacetate=3:7). ethylacetate was first used to precipitate out pale yellowish white crystals, then ethanol:ethylacetate=1:9 was used to repeatedly re-crystallized to obtain purified compound 5.

The structure of compound obtained, compound 5 is C₂₆H₃₇O₆N₂Cl, and the molecular weight through mass spectrometer is 508.5. ¹H-NMR(CDCl₃) δ:1.11-1.22(m, 7H,N—CH₂CH₂CH₂CH₃), 1.22-1.29(m, 6H, 2×CO—OCH₂CH₃), 2.04(s, 6H, 2×CH₃), 2.17-2.71(m, 4H, CH₂—NH—CH₂), 3.4 (m, 4H,2×CO—OCH₂CH₃), 3.97-4.14(m, 2H, Ar—OCH₂), 4.3-4.5(m, 1H,CH—OH), 5.33(s, 1H, Ar—CH<), 6.65-7.27(m, 3H, Ar). MS m/s: 508.5(Scan FAB+) Anal. (C₂₆H₃₇O₆N₂Cl)C,H,N. In accordance to the analytical data obtained from chemical experiments, compound 5 was found to be 4-{{2-[2-hydroxy-3-(n-butylamine)propoxy]-5-choloro}phenyl)}-2,6-dimethyl-3,5-dicarbo-e thoxy-1,4-dihydropyridine.

### EXAMPLE 6

### Synthesis of Compound 6

8 gm of sodium hydroxide was dissolved in 100 ml absolute alcohol. 1 molar of 5-chlorosalicylaldehyde was dissolved in the above mentioned solution. This solution was stirred under room temperature, and 5 molar of epichlorohydrin added to react. TLC was used to determine that the reaction had been completed. After decompressed to concentrate, the solution was separated by silica gel filled column, with hexane: ethylacetate=1:9 as the eluent solution. A white coarse crystal was obtained. With repeated re-crystallization by hexane to obtain purified N-[2-(2,3-epoxypropoxy)-5-chloro]-1-benzaldehyde.

Similar molar of N-[2-(2,3-epoxypropoxy)-5-chloro]-1-benzaldehyde and 2-methoxy-1-oxyethylamiao-benzene were dissolved in 100 ml of absolute, and undergone amination in slight warmth. Using column chromatography (methanol:ethylacetate=1:1)to purify, a white solid crystal could be obtained. Using methanol to re-crystallized, purified compound N-{2-[2-hydroxy-3-(2-methoxy-1-oxyethylamio-benzene)propoxy]-5-chloro}-1-benzaldehyde was obtained.

0.01M of N-{2-[2-hydroxy-3-(2-methoxy-1-oxyethylamio-benzene)propoxy]-5-chloro}-1-benzaldehyde was dissolved in a solution containing 2.4 ml (0.02M) ethylacetoacetate, 15 ml ethanol and 10 ml concentrated ammonia water; heated and placed in a water bath of 55° C. for 15 hours. After the reaction, the solution obtained was directly decompressed to concentrate and ethanol removed. 50 ml of saturated NaCO₃ solution was added to the remaining solution and the organic layers were extracted by CHCl₃ and water for several times. All the organic layers obtained were dehydrated; filtered and concentrated, then the oil layer obtained was added to a Ethanol-HCl mixed solution, and purified by column chromatography (methanol:ethylacetate=3:7). Ethylacetate was first used to precipitate out pale yellowish white crystals, then ethanol:ethylacetate=1:9 was used to repeatedly re-crystallized to obtain purified compound 6.

The structure of compound obtained, compound 6 is C₃₁H₃₉O₈N₂Cl, and the molecular weight through mass spectrometer is 602.5. ¹H-NMR(CDCl₃) δ:1.17-1.28(m, 6H, 2×CO—OCH₂CH₃), 2.27-2.29(m, 6H, 2×CH₃), 2.50-2.89(m, 4H, CH₂—NH—CH₂), 3.67-3.77(m, 4H, 2×CO—OCH₂CH₃), 3.84 (s, 3H, OCH₃), 3.97-4.14(m, 4H, 2×Ar—OCH₂), 4.26-4.33(m, 1H,CH—OH), 5.3 (s, 1H, Ar—CH<), 5.6 (brs, 1H, replaceable, —NH—). MS m/s: 602.5(Scan FAB+) Anal. (C₃₁H₃₉O₈N₂C1)C,H,N. In accordance to the analytical data obtained from chemical experiments, compound 6 was found to be 4-{{2-[2-hydroxy-3-(2-methoxy-1-oxyethylamino-benzene)propoxy]-5-choloro}phenyl}}-2, 6-dimethyl-3,5-dicarbo-ethoxy-1,4-dihydropyridine.

### EXAMPLE 7

### Synthesis of Compound 7

8 gm of sodium hydroxide was dissolved in 100 ml absolute alcohol. 1 molar of 2-chloro-4-hydroxy-benzaldehyde was dissolved in the above mentioned solution. This solution was stirred under room temperature, and 5 molar of epichlorohydrin added to react. TLC was used to determine that the reaction had been completed. After decompressed to concentrate, the solution was separated by silica gel column, with hexane: ethylacetate=1:9 as the eluent solution. A white coarse crystal was obtained. With repeated re-crystallization by hexane to obtain purified N-[4-(2,3-epoxypropoxy)-2-chloro]-1-benzaldehyde.

Similar molar of N-[4-(2,3-epoxypropoxy)-2-chloro]-1-benzaldehyde and 2-methoxy-1-oxyethylaminobenzene were dissolved in 100 ml of absolute alcohol, and undergone amination in slight warmth. After stirring and left to rest overnight, a white solid crystal could be obtained. Using methanol to re-crystallized, purified compound N-{4-[2-hydroxy-3-(2-methoxy-1-oxyethyl-aminobenzene)propoxy]-2-chloro}-1-benzaldehyde was obtained.

0.01M of N-{4-[2-hydroxy-3-(2-methoxy-1-oxyethyl-aminobenzene)propoxy]-2-chloro}-1-benzaldehyde was dissolved in a solution containing 2.4 ml (0.02M) ethylacetoacetate, 15 ml ethanol and 10 ml concentrated ammonia water; heated and placed in a water bath of 55° C. for 15 hours. After the reaction, the solution obtained was directly decompressed to concentrate and ethanol removed. 50 ml of saturated NaCO₃ solution was added to the remaining solution and the organic layers were extracted by CHCl₃ for several times. All the organic layers obtained were dehydrated; filtered and concentrated, then the oil layer obtained was added to a Ethanol-HCl mixed solution, and purified by column chromatography (methanol:ethylacetate=3:7). Ethylacetate was first used to precipitate out pale yellowish white crystals, then ethanol: ethylacetate=1:9 was used to repeatedly re-crystallized to obtain purified compound 7.

The structure of compound obtained, compound 7 is C₃₁H₃₉O₈N₂C1, and the molecular weight through mass spectrometer is 602.5. ¹H-NMR(CDCl₃) δ:1.21-1.29(m, 6H, 2×CO—OCH₂CH₃), 2.04(m, 6H, 2×CH₃), 2.29-2.50(m, 4H, CH₂—NH—CH₂), 3.82-3.86(m, 4H, 2×CO—OCH₂CH₃), 3.87-3.98 (s, 3H, OCH₃), 4.06-4.24(m, 4H, 2×Ar— OCH₂), 4.6(m, 1H,CH—OH), 5.31 (s, 1H, Ar—CH<), 5.75(brs, 1H, replaceable, —NH—). MS m/s: 602.5(Scan FAB+) Anal. (C₃₁H₃₉O₈N₂Cl)C,H,N. In accordance to the analytical data obtained from chemical experiments, compound 7 was found to be 4-{{N-{4-[2-hydroxy-3-(2-methoxy-1-oxyethyl-aminobenzene)propoxy]-3-choloro}phenyl} }-2,6-dimethyl-3,5-dicarbo-ethoxy-1,4-dihydropyridine.

### EXAMPLE 8

### Synthesis of Compound 8

8 gm of sodium hydroxide was dissolved in 100 ml absolute alcohol. 1 molar of 5-nitrosalicylaldehyde was dissolved in the NaOH solution described as above. This solution was stirred under room temperature, and 5 molar of epichlorohydrin added to react. TLC was used to determine that the reaction had been completed. After decompressed to concentrate, the solution was separated by silica gel column, with hexane: ethylacetate=1:9 as the eluent solution. A white coarse crystal was obtained. With repeated re-crystallization by hexane to obtain purified N-[2-(2,3-epoxypropoxy)-5-nitro]-1-benzaldehyde.

Similar molar of N-[2-(2,3-epoxypropoxy)-5-nitro]-1-benzaldehyde and tert-butylamine were dissolved in 100 ml of absolute alcohol, and undergone amination in slight warmth. Using column chromatography (methanol:ethylacetate=1:1) to purify, a white solid crystal could be obtained. Using methanol to re-crystallized, purified compound N-{2-[2-hydroxy-3-(tert-butylamino)propoxy]-5-nitro}-1-benzaldehyde was obtained.

0.01M of N-{2-[2-hydroxy-3-(tert-butylamino)propoxy]-5-nitro}-1-benzaldehyde was dissolved in a solution containing 2.4 ml (0.02M) ethylacetoacetate, 15 ml ethanol and 10 ml concentrated ammonia water; heated and placed in a water bath of 55° C. for 15 hours. After the reaction, the solution obtained was directly decompressed to concentrate and ethanol removed. 50 ml of saturated NaCO₃ solution was added to the remaining solution and the organic layers were extracted by CHCl₃ and water for several times. All the organic layers obtained were dehydrated; filtered and concentrated, then the oil layer obtained was added to a Ethanol-HCl mixed solution, and purified by column chromatography (methanol:ethylacetate=3:7). Ethylacetate was first used to precipitate out pale yellowish white crystals, then ethanol ethylacetate=1:9 was used to repeatedly re-crystallized to obtain purified compound 8.

The structure of compound obtained, compound 8 is C₂₆H₃₇O₈N₃ and the molecular weight through mass spectrometer is 519. ¹H-NMR(CDCl₃) δ:1.14-1.16(s, 9H,3xCH₃), 1.17-1.24(m, 6H, 2×CO—OCH₂CH₃), 2.05(d, 6H, 2xCH₃), 2.33-2.34(m, 4H, CH2—NH), 2.85-2.98 (s, 4H, 2×CO—OCH₂CH₃), 3.98-4.17(m, 2H, Ar—OCH₂), 4.24-4.26(s,1H,CH—OH), 5.39 (s, 1H, Ar—CH<), 6.82-8.14(m, 3H, Ar). MS m/s: 519(Scan FAB+) Anal. (C₂₆H₃₇O₈N₃)C,H,N. In accordance to the analytical data, compound 8 was found to be 4-{{2-[2-hydroxy-3-(tert-butylamino)propoxy]-5-nitro} phenyl)}-2,6-dimethyl-3,5-dicarbo-et hoxy-1,4-dihydropyridine.

### EXAMPLE 9

### Synthesis of Compound 9

8 gm of sodium hydroxide was dissolved in 100 ml absolute alcohol. 1 molar of 5-nitrosalicylaldehyde was dissolved in the above mentioned solution. This solution was stirred under room temperature, and 5 molar of epichlorohydrin added to react. TLC was used to determine that the reaction had been completed. After decompressed to concentrate, the solution was separated by silica gel column, with hexane: ethylacetate=1:9 as the eluent solution. A white coarse crystal was obtained. With repeated re-crystallization by hexane to obtain purified N-[2-(2,3-epoxypropoxy)-5-nitro]-1-benzaldehyde.

Similar molar of N-[2-(2,3-epoxypropoxy)-5-nitro]-1-benzaldehyde and 2-methoxy-1-oxyethylamino benzene were dissolved in 100 ml of absolute alcohol, and undergone amination in slight warmth. After stirring and left to rest overnight, a white solid crystal could be obtained. Using methanol to re-crystallized, purified compound N-{2-[2-hydroxy-3-(2-methoxy-1-oxyethyl-aminobenzene)propoxy]-5-nitro}-1-benzaldehyde was obtained.

0.01M of N-{2-[2-hydroxy-3-(2-methoxy-1-oxyethylamino benzene)propoxy]-5-nitro}-1-benzaldehyde was dissolved in a solution containing 2.4 ml (0.02M) ethylacetoacetate, 15 ml ethanol and 10 ml concentrated ammonia water; heated and placed in a water bath of 55° C. for 15 hours. After the reaction, the solution obtained was directly decompressed to concentrate and ethanol removed. 50 ml of saturated NaCO₃ solution was added to the remaining solution and the organic layers were extracted by CHCl₃ and water for several times. All the organic layers obtained were dehydrated; filtered and concentrated, then the oil layer obtained was added to a Ethanol-HCl mixed solution, and purified by column chromatography (methanol:ethylacetate=3:7). Ethylacetate was first used to precipitate out pale yellowish white crystals, then ethanol:ethylacetate=1:9 was used to repeatedly re-crystallized to obtain purified compound 9.

The structure of compound obtained, compound 9 is C₃₁H₃₅O₁₀N₃ and the molecular weight through mass spectrometer is 609. ¹H-NMR(CDCl₃) δ:1.14-1.28(m, 6H, 2×CO—OCH₂CH₃), 2.04(m, 6H, 2xCH₃), 2.22-2.33(m, 4H, CH₂—NH—CH₂), 3.40-3.60(m, 4H, 2×CO—OCH₂CH₃), 3.83-3.85 (s, 3H, OCH₃), 3.99-4.13(m, 4H, 2×Ar—OCH₂), 4.3(m,1H,CH—OH), 5.41 (s, 1H, Ar—CH<), 6.5(brs, 1H, replaceable, —NH—), 6.82-8.12(m, 7H,Ar). MS m/s: 609(Scan FAB⁺) Anal. (C₃₁H₃₅O₁₀N₃)C,H,N. In accordance to the analytical data obtained from chemical experiments, compound 9 was found to be 4-{{2-[2-hydroxy-3-(2-methoxy-1-oxyethylaminobenzene)propoxy]-5-nitro}phenyl}}-2,6-di methyl-3,5-dicarbo-ethoxy-1,4-dihydropyridine.

### EXAMPLE 10

### Synthesis of Compound10

8 gm of sodium hydroxide was dissolved in 100 ml absolute alcohol. 1 molar of 4-hydroxy-3-methoxy-1-benzaldehyde was dissolved in NaOH solution mentioned on the above. This solution was stirred under room temperature, and according with example 3, 5 molar of epichlorohydrin was added to react under room temperature. TLC was used to determine that the reaction had been completed. Similar procedure as the example 3 mentioned, purified compound 10 will be obtained.

The structure of compound obtained, compound 10 is C₃₂H₄₂O₉N₂ and the molecular weight through mass spectrometer is 598. Compound 10 was found to be 4-{{N-{3-[2-hydroxy-3-(2-methoxy-1-oxyethylaminobenzene)propoxy]-4-methoxy} phenyl}}-2,6-dimethyl-3,5-dicarbo-ethoxy-1,4-dihydropyridine.

TABLE I

| | Lowering | Reduction of Heart | Aorta |
|---|---|---|---|
| | of Blood Pressure | Rate | Relaxation |
| Compound | (1 mg/kg; mmHg) | (1 mg/kg; beats/min) | (10⁻⁸M; %) |
| 1 | 70 ± 3 | 30 ± 2 | 30 ± 3 |
| 2 | 75 ± 2 | 35 ± 4 | 40 ± 4 |
| 3 | 78 ± 5 | 37 ± 5 | 41 ± 2 |
| 4 | 76 ± 6 | 33 ± 3 | 35 ± 5 |
| 5 | 74 ± 4 | 32 ± 4 | 33 ± 2 |
| 6 | 80 ± 3 | 35 ± 5 | 45 ± 4 |
| 7 | 82 ± 6 | 40 ± 2 | 45 ± 5 |
| 8 | 80 ± 3 | 40 ± 4 | 43 ± 3 |
| 9 | 85 ± 6 | 40 ± 5 | 47 ± 2 |

| | | β₁ | | | | |
|---|---|---|---|---|---|---|
| | Calcium | Value of pA₂ | | β₂ | | |
| | Antagonism | Right Atrium | Left Atrium | Value of pA₂ | Ratio of β₁ and | |
| Compound | PKCa⁻¹ | (Slope) | (Slope) | Tracheal (slope) | Calcium Antagonism | β₁/β₂ |
| 1 | 7.82 ± 0.49 | 7.21 ± 0.32 | 6.91 ± 0.26 | 7.09 ± 0.54 | 0.12 | 1.32 |
| | | (0.94 ± 0.18) | (0.85 ± 0.02) | (0.84 ± 0.09) | | |
| 2 | 7.91 ± 0.35 | 7.36 ± 0.65 | 6.93 ± 0.34 | 7.20 ± 0.45 | 0.09 | 1.28 |
| | | (0.88 ± 0.09) | (0.91 ± 0.21) | (0.88 ± 0.12) | | |
| 3 | 7.96 ± 0.27 | 7.47 ± 0.38 | 6.97 ± 0.12 | 7.31 ± 0.23 | 0.08 | 1.26 |
| | | (0.92 ± 0.27) | (0.89 ± 0.03) | (0.92 ± 0.23) | | |
| 4 | 7.94 ± 0.56 | 7.38 ± 0.12 | 6.90 ± 0.35 | 7.27 ± 0.32 | 0.08 | 1.31 |
| | | (0.83 ± 0.12) | (0.92 ± 0.12) | (0.87 ± 0.18) | | |
| 5 | 7.89 ± 0.34 | 7.37 ± 0.27 | 6.91 ± 0.09 | 7.24 ± 0.12 | 0.10 | 1.33 |
| | | (0.91 ± 0.23) | (0.93 ± 0.19) | (0.91 ± 0.21) | | |
| 6 | 8.01 ± 0.45 | 7.41 ± 0.34 | 7.01 ± 0.28 | 7.31 ± 0.19 | 0.11 | 1.29 |
| | | (0.88 ± 0.12) | (0.91 ± 0.09) | (0.90 ± 0.19) | | |
| 7 | 8.06 ± 0.27 | 7.77 ± 0.34 | 6.97 ± 0.29 | 7.28 ± 0.21 | 0.07 | 1.34 |
| | | (0.91 ± 0.08) | (0.88 ± 0.27) | (0.89 ± 0.21) | | |
| 8 | 8.09 ± 0.13 | 7.89 ± 0.23 | 7.03 ± 0.18 | 7.27 ± 0.19 | 0.08 | 1.29 |
| | | (0.83 ± 0.21) | (0.89 ± 0.12) | (0.92 ± 0.17) | | |
| 9 | 8.12 ± 0.32 | 7.78 ± 0.12 | 7.12 ± 0.09 | 7.33 ± 0.24 | 0.07 | 1.30 |
| | | (0.88 ± 0.09) | (0.91 ± 0.18) | (0.88 ± 0.23) | | |
| Propranolol | Not tested | 8.32 ± 0.06 | 8.23 ± 0.09 | 8.09 ± 0.12 | | 1.7 |
| | | (0.95 ± 0.04) | (0.81 ± 0.05) | (0.95 ± 0.08) | | |

TABLE 3

| | [³H]nitrendipine | [³H] CGP-12177 | [³H] CGP-12177 |
|---|---|---|---|
| | Ca²⁺ | (β₁) | (β₂) |
| Compound | PKi | pKi | pKi |
| I | 7.86 ± 0.38 | 6.61 ± 0.46 | 6.20 ± 0.55 |
| Nifedipine | 8.70 ± 0.25 | NT | NT |
| Propranolol | NT | 9.12 ± 0.14 | 8.61 ± 0.1 |

1. A compound of formula Ia: 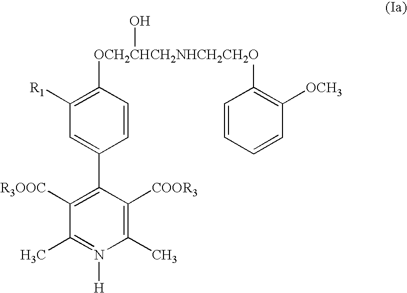
wherein R₁ and R₃ are each individually selected from the group consisting of —X, —H, —NO₂, CF₃, saturated C₁-C₆ alkyl chain, unsaturated C₂-C₆ alkyl chain, saturated C₁-C₆ alkoxy chain, and unsaturated C₂-C₆ alkoxy chain, and wherein X represents a halogen.

2. A pharmaceutical composition that comprises an effective amount of the compound of formula Ia of claim 1 and a pharmaceutically acceptable carrier, diluent or excipient.

3. A method for blocking an α-, β-adrenoreceptor or calcium channel that comprises administering to a patient in need thereof an effective amount of the composition of claim 2.

4. A method for inducing hypotension that comprises administering to a patient in need thereof an effective amount of the composition of claim 2.

5. A method for inducing vaso-relaxation that comprises administering to a patient in need thereof an effective amount of the composition of claim 2.

6. A compound of formula Ib: 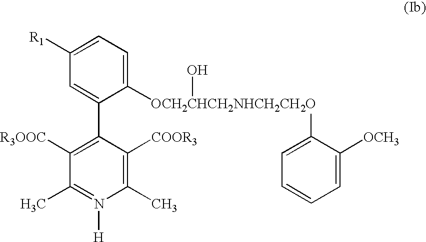
wherein R₁ and R₃ are each individually selected from the group consisting of —X, —H, —NO₂, CF₃, saturated C₁-C₆ alkyl chain, unsaturated C₂-C₆ alkyl chain, saturated C₁-C₆ alkoxy chain, and unsaturated C₂-C₆ alkoxy chain, and wherein X represents a halogen.

7. A pharmaceutical composition that comprises an effective amount of the compound of formula Ib of claim 6 and a pharmaceutically acceptable carrier, diluent or excipient.

8. A method for blocking an α-, β-adrenoreceptor or calcium channel that comprises administering to a patient in need thereof an effective amount of the composition of claim 7.

9. A method for inducing hypotension that comprises administering to a patient in need thereof an effective amount of the composition of claim 7.

10. A method for inducing vaso-relaxation that comprises administering to a patient in need thereof an effective amount of the composition of claim 7.
